(19) United States
(12) Patent Application Publication
(10) Pub. No.: US 2026/0120828 A1
(43) Pub. Date: Apr. 30, 2026

(54) Generating Explanations Based On User Knowledge And/Or Familiarity With A Target Topic

(71) Applicant: Oracle International Corporation, Redwood Shores, CA (US)

(72) Inventor: Jennifer Darmour, Seattle, WA (US)

(73) Assignee: Oracle International Corporation, Redwood Shores, CA (US)

(21) Appl. No.: 19/370,118

(22) Filed: Oct. 27, 2025

Related U.S. Application Data
(60) Provisional application No. 63/712,905, filed on Oct. 28, 2024.

(51) Int. Cl. G16H 10/60

(52) U.S. Cl. CPC G16H 10/60

(57) ABSTRACT

Techniques for generating knowledge-adapted content based on a knowledge classification of a user are disclosed. A system determines that a trigger condition is satisfied for requesting a knowledge-adapted content element for augmenting information for display on a user interface. In response to determining that the trigger condition is satisfied, the system generates, in real time, an input prompt element for requesting the knowledge-adapted content element and directs the input prompt element to a machine learning (ML) model to generate the knowledge-adapted content element. The knowledge-adapted content element includes machine-generated content pertaining to the target concept. The system receives the knowledge-adapted content element from the ML model and augments the information at least by concurrently displaying the information and machine-generated content on the user interface.

### BENEFIT CLAIMS; RELATED APPLICATIONS; INCORPORATION BY REFERENCE

This application claims the benefit of U.S. Provisional Patent Application 63/712,905, filed Oct. 28, 2024, that is hereby incorporated by reference.

The Applicant hereby rescinds any disclaimer of claim scope in the parent application(s) or the prosecution history thereof and advises the USPTO that the claims in this application may be broader than any claim in the parent application(s).

### TECHNICAL FIELD

The present disclosure relates to generating explanations of information. More particularly, the present disclosure relates to utilizing machine learning (ML) models to generate information.

### BACKGROUND

A person may access information by interacting with a user interface. For example, when a patient receives care from a healthcare provider, electronic health records (EHRs) are generated that include health information pertaining to the patient. A patient may access their health information by interacting with a user interface. The user interface displays various health information for the patient. Likewise, a person may access other types of information by interacting with a user interface.

A person is more likely to understand information about a topic when the information is tailored to the level of knowledge and/or familiarity of the person with the topic. For example, healthcare providers want patients to review and understand their health information. Patients are more likely to review their health information, and patients are more likely to understand the health information that they review when their health information is presented in a context that aligns with the level of knowledge and/or familiarity of the patient. Conversely, patients may ignore health information if they do not understand it and/or if the information lacks contextual relevance to the patient. Healthcare providers also want patients to proactively manage their health, including acting in response to their health information. As examples, this may include self-care, such as diet and exercise, taking medications as prescribed, and/or scheduling and attending healthcare appointments. Patients are more likely to proactively manage their health when their health information is explained in a way that is understandable and contextually relevant to the patient.

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

### BRIEF DESCRIPTION OF THE DRAWINGS

The embodiments are illustrated by way of example and not by way of limitation in the figures of the accompanying drawings. It should be noted that references to “an” or “one” embodiment in this disclosure are not necessarily to the same embodiment, and they mean at least one. In the drawings:

FIG. 1 is a block diagram that depicts an example computing architecture for a system in accordance with one or more embodiments;

FIGS. 2A-2C illustrate example operations associated with utilizing ML models to generate explanations of information for a person based on a level of knowledge and/or familiarity of the person with a target topic in accordance with one or more embodiments;

FIGS. 3A-3E show an example user interface that includes explanations of information generated for a person by an ML model based on a level of knowledge and/or familiarity of the person with a target topic in accordance with one or more embodiments;

FIG. 4 illustrates features of an example ML system in accordance with one or more embodiments;

FIG. 5 is a flowchart that depicts example operations of an ML system in accordance with one or more embodiments; and

FIG. 6 shows a block diagram that illustrates a computer system in accordance with one or more embodiments.

### DETAILED DESCRIPTION

In the following description, for the purposes of explanation, numerous specific details are set forth to provide a thorough understanding. One or more embodiments may be practiced without these specific details. Features described in one embodiment may be combined with features described in a different embodiment. In some examples, well-known structures and devices are described with reference to a block diagram form to avoid unnecessarily obscuring the present disclosure.
1. GENERAL OVERVIEW
2. EXAMPLE SYSTEM ARCHITECTURE FOR GENERATING EXPLANATIONS OF TARGET CONCEPTS
3. EXAMPLE OPERATIONS PERTAINING TO GENERATING EXPLANATIONS OF TARGET CONCEPTS
4. EXAMPLE USER INTERFACES
5. PRACTICAL APPLICATIONS, ADVANTAGES, AND IMPROVEMENTS
6. EXAMPLE MACHINE LEARNING SYSTEM
7. COMPUTER NETWORKS AND CLOUD NETWORKS
8. HARDWARE OVERVIEW
9. MISCELLANEOUS; EXTENSIONS

### 1. GENERAL OVERVIEW

One or more embodiments adapt information to be presented to a user based on the user's knowledge of a topic(s) associated with the information. The system generates knowledge-adapted content based on a knowledge classification of a user, to augment a set of information that is to be displayed to a user. Initially, the system determines that a trigger condition is satisfied for requesting a knowledge-adapted content element. The system utilizes the knowledge-adapted content element to augment information for display on a user interface based on a knowledge classification of the user with respect to a target concept associated with the information. In response to determining that the trigger condition is satisfied, the system generates, in real time, an input prompt element for requesting the knowledge-adapted content element. The system directs the input prompt element to an ML model to generate the knowledge-adapted content element. The knowledge-adapted content element includes machine-generated content pertaining to the target concept. The system receives the knowledge-adapted content element from the ML model and augments the information for display on the user interface. In one example, the system augments the information for display on the user interface by concurrently displaying the information and machine-generated content on the user interface.

In one example, the system predicts a user comprehension of a target concept and then generates a natural language explanation of the target concept based on the predicted user comprehension of the target concept. The system may generate a natural language explanation of the predicted user comprehension of the target concept and utilize the natural language explanation of the predicted user comprehension as an input for generating the natural language explanation of the target concept. The system may generate the natural language explanation of the target concept based on grounding information corresponding to the knowledge classification of the user.

One or more embodiments generate adaptive query responses that include knowledge-adapted content suitable for a user based on the user's knowledge base and/or experiences. Adaptive query responses include query responses specifically adapted for a target user for improved understanding by the target user. The system predicts a depth of knowledge or a user's familiarity with a target topic based on, for example, contextual information associated with the user. The contextual information may include experiences associated with the target topic. The contextual information may include historical user data associated with the target topic. The system applies an ML model to a prompt that includes both a query and the user's contextual information to generate a response. In one example, the system generates general, high-level descriptions/explanations in response to a query for symptoms by a patient that is beginning a medical treatment. The system generates detailed, low-level descriptions/explanations in response to a query of symptoms by a patient that has been going through medical treatments for a significant portion of time.

One or more embodiments utilize an ML model to generate knowledge-adapted content. The knowledge-adapted content may include an explanation of a target concept for a person based on the person's level of knowledge and/or familiarity with the target concept. The explanation of the target concept is displayed on a user interface to explain the target concept in a way that is tailored to the knowledge level and/or familiarity of the person. The ML model may include and/or exclude content from the explanation based on the knowledge level and/or familiarity of the user. Additionally, or alternatively, the ML model may provide a level of detail based on the knowledge level and/or familiarity of the user. Additionally, or alternatively, the ML model may generate the explanation utilizing a communication style determined by the ML model based on the knowledge level and/or familiarity of the user with respect to the target concept.

In one example, the system detects an occurrence of a trigger event and determines whether the trigger event satisfies one or more trigger conditions for generating knowledge-adapted content, such as an explanation of a target concept, for display on a user interface. The trigger event may include an interaction with the user interface and/or updates to information in electronic records. The system analyzes the trigger event against the one or more trigger conditions. The one or more trigger conditions include a set of one or more rules, criteria, or conditions that define when the system will generate an explanation of a target concept. When a trigger event satisfies a trigger condition, the system utilizes an ML model to predict a level of knowledge and/or familiarity of the user with respect to a target concept. Additionally, or alternatively, the system utilizes an ML model to generate the knowledge-adapted content, such as the explanation of the target concept, based on the knowledge level and/or familiarity of the user with respect to the target concept. The ML model may include and/or exclude content from the explanation based on the contextual information. Additionally, or alternatively, the ML model may generate the explanation utilizing a communication style that is determined by the ML model based on the contextual information.

One or more embodiments described in this Specification and/or recited in the claims may not be included in this General Overview section.

### 2. EXAMPLE SYSTEM ARCHITECTURE FOR GENERATING EXPLANATIONS OF TARGET CONCEPTS

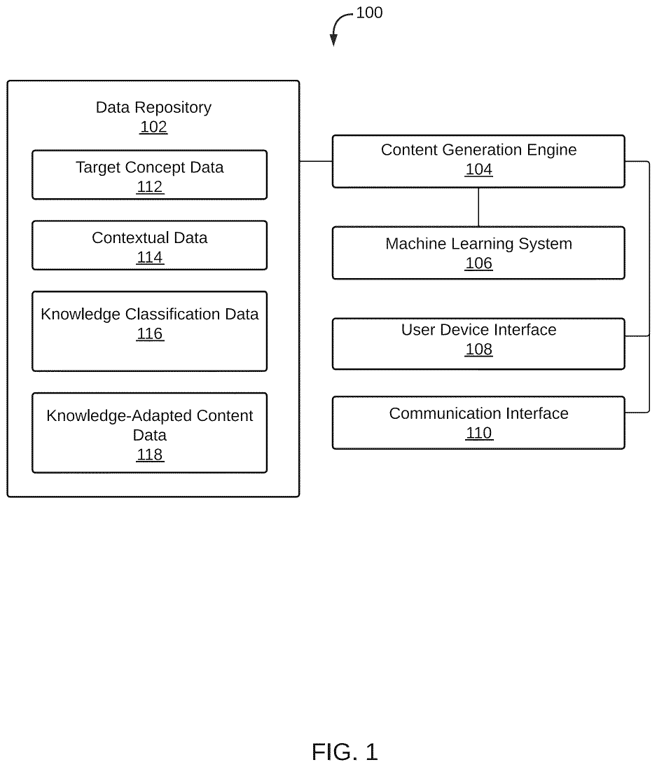

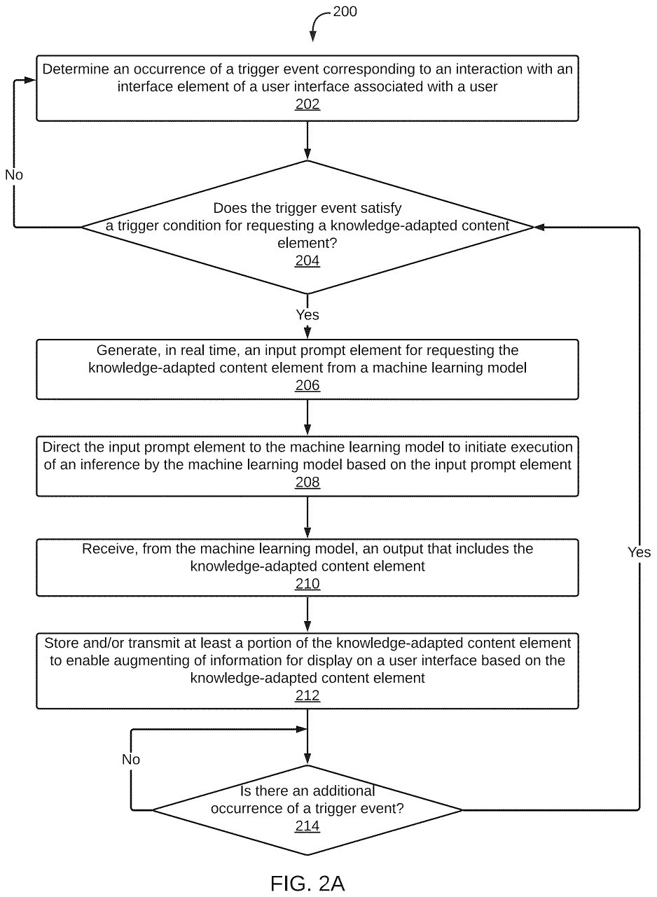

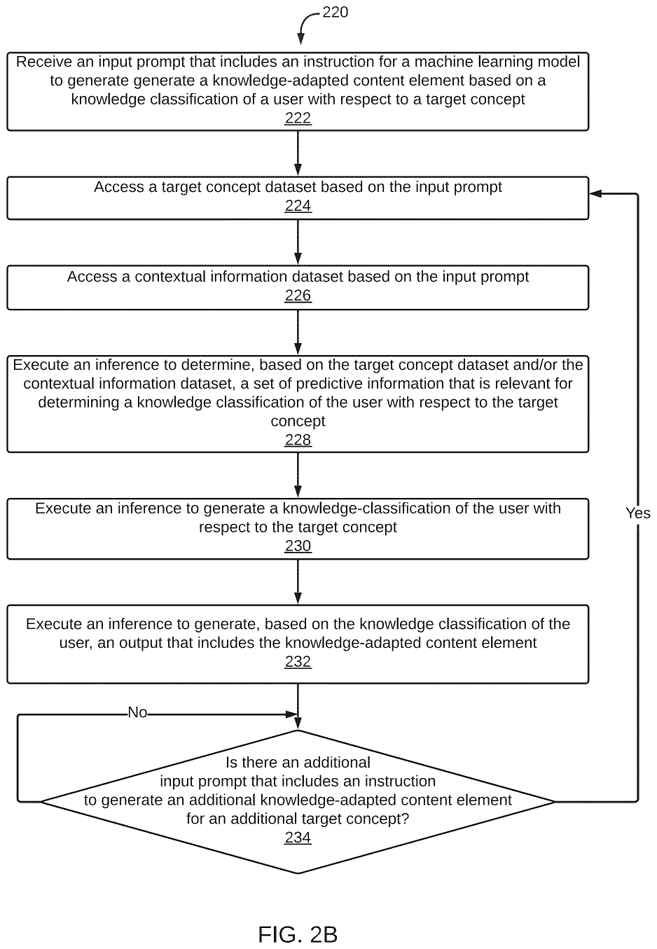

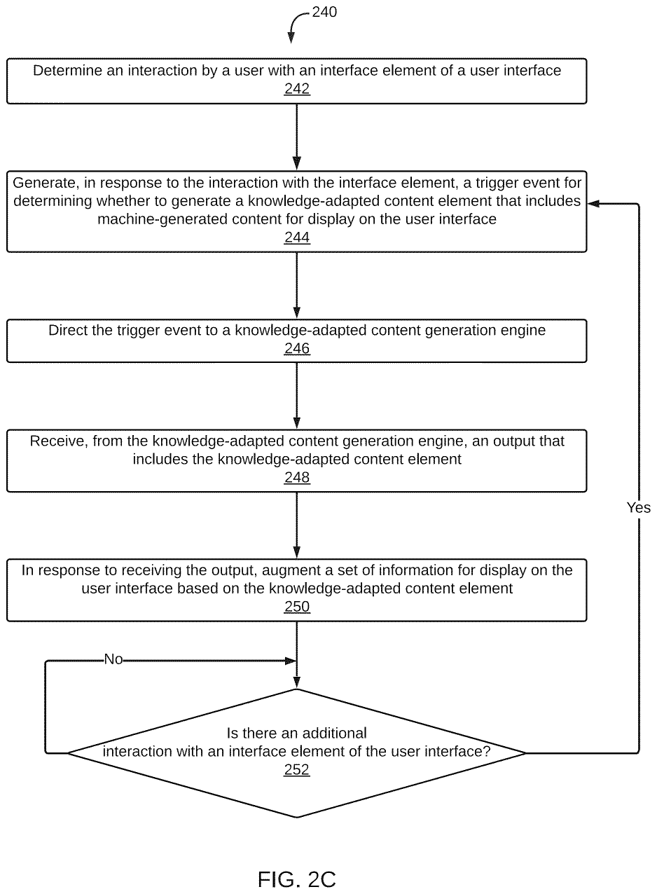

FIG. 1 illustrates features of an example system 100 in accordance with one or more embodiments. The system 100 may execute operations associated with generating knowledge-adapted content elements based on a knowledge classification of a user with respect to a target concept and utilizing the knowledge-adapted content elements to augment information for display on a user interface. The knowledge-adapted content elements include machine-generated content generated by an ML model based on the knowledge classification of the user with respect to the target concept. The machine-generated content may include explanations of target concepts. In one example, the target concepts are health concepts, for example, pertaining to information in EHRs associated with a patient. In one or more embodiments, the system 100 refers to hardware and/or software configured to perform operations described herein. Examples of operations are described below with reference to FIGS. 2A-2C.

As shown in FIG. 1, the system 100 includes a data repository 102, a content generation engine 104, an ML system 106, a user device interface 108, and a communication interface 110. The data repository 102 includes data utilized by the content generation engine 104 to execute operations described herein. Additionally, or alternatively, the data repository 102 may include data generated or obtained by the content generation engine 104 when executing operations described herein. The content generation engine 104 executes operations associated with generating knowledge-adapted content based on a knowledge classification of a user with respect to a target concept.

In one or more embodiments, the data repository 102 includes any type of storage unit and/or device (e.g., a file system, database, collection of tables, or any other storage mechanism) for storing data. Furthermore, the data repository 102 may include multiple different storage units and/or devices. The multiple different storage units and/or devices may or may not be of the same type or located at the same physical site. Furthermore, the data repository 102 may be implemented or executed on the same computing system as the content generation engine 104. Additionally, or alternatively, the data repository 102 may be implemented or executed on a computing system separate from the content generation engine 104. The data repository 102 may be communicatively coupled to the content generation engine 104 via a direct connection or via a network.

As shown in FIG. 1, the data repository 102 may include one or more of the following: target concept data 112, contextual data 114, knowledge classification data 116, and knowledge-adapted content data 118. The target concept data 112 includes data pertaining to one or more target concepts. In one example, the target concept data 112 may include health data. The health data may be stored in EHRs. The contextual data 114 includes data that provides contextual information about different users. The contextual data may represent a portion of the target concept data 112 and/or data that is not target concept data 112. The knowledge classification data 116 includes data that classifies one or more attributes pertaining to the knowledge of a user with respect to a target concept. The knowledge classification data 116 may describe a level of knowledge and/or familiarity of a user with respect to a target concept such as a health concept. The knowledge-adapted content data 118 includes knowledge-adapted content elements that include machine-generated data generated by the ML system 106 based on a knowledge classification of a user with respect to a target concept.

The content generation engine 104 directs input prompts to the ML system 106 that include instructions for the ML system 106 to generate a knowledge-adapted content element based on a knowledge classification of the user with respect to a target concept. The knowledge-adapted content element may include an explanation of a target concept based on a user's level of knowledge and/or familiarity with the target concept. In one example, the ML system 106 predicts a user comprehension of a target concept and then generates a natural language explanation of the target concept based on the predicted user comprehension of the target concept. Additionally, or alternatively, the ML system 106 may generate a knowledge classification that includes a natural language explanation of the predicted user comprehension of the target concept. The ML system may utilize the natural language explanation of the predicted user comprehension as an input to generate the natural language explanation of the target concept.

The input prompts may be generated in response to trigger events, such as interactions with a user device interface 108 and/or updated information being added to electronic records associated with a user. The ML system 106 includes ML algorithms executed by the ML system 106 to generate outputs that include the knowledge-adapted content element. The ML system 106 provides the outputs to the content generation engine 104, and the content generation engine 104 stores and/or transmits at least a portion of the outputs to enable the user device interface 108 to display machine-generated content from the knowledge-adapted content element.

The ML system 106 may predict a knowledge classification of a user with respect to a target concept such as a health concept. The knowledge classification may represent a level of knowledge and/or familiarity of a user with respect to the target concept. The ML system 106 may predict the knowledge classification based on contextual information. Additionally, or alternatively, the ML system 106 may generate a knowledge-adapted content element based on the knowledge classification of the user. The knowledge-adapted content element may include machine-generated content, such as an explanation of the target concept, based on the level of knowledge and/or familiarity of the user with the target concept. The ML system 106 may include and/or exclude content from the machine-generated content based on the level of knowledge and/or familiarity of the user with the target concept. Additionally, or alternatively, the ML system 106 may generate the machine-generated content utilizing a communication style determined by the ML system 106 based on the level of knowledge and/or familiarity of the user with the target concept. The communication style may include one or more of the following aspects that are based on the contextual information: wording, tone, clarity, pacing, emotion, emphasis, formality, intention, structure, consistency, grammar, punctuation, syntax, word choice, formatting, length, conciseness, or directness. An example ML system is further described below in Section 6, titled “Example Machine Learning System.” The ML system may perform one or more operations described with reference to FIGS. 2A-2C.

The term “target concept” refers to a concept that may be of interest to a user, for example, that is interacting with a user interface. The target concept may include one or more items of target concept data 112, information generated based on target concept data 112, and information generated by combining target concept data 112 with other sources of information.

The term “health concept” refers to an abstract or specific idea, notion, or element related to one or more aspects of a person's health. An aspect of a person's health may include physical health, mental health, social health, occupational health, and/or environmental health. An aspect of a person's health may include medical conditions, diagnoses, treatments, preventative measures, wellness practices, and/or health-related behaviors. Additionally, or alternatively, an aspect of a persons'health may include symptoms, disease management, medical interventions, risk factors, and/or outcomes associated with the person's health. Additionally, or alternatively, an aspect of a person's health may include one or more items of health data. Health concepts may be utilized in healthcare, medical research, and/or patient care to organize and communicate health-related information.

The term “health data” refers to data directly or indirectly associated with the health or healthcare of a person. As examples, health data may include data associated with one or more of the following: medical history, diagnoses, conditions, treatments, visits, appointments, clinical notes, surgeries, radiology images, test results, medications, allergies, medical device information, mental health conditions, mental health treatments, physical therapy treatments, occupational therapy treatments, genetic information, personal habits, sexual health information, immunizations, health insurance information, vital signs, biometric information, disability status, accommodations, substance use history, reproductive information, physical fitness information, or family medical history.

The term “contextual information” refers to information pertaining to circumstances, conditions, or factors surrounding an event, statement, person, or idea that help clarify its meaning or significance. Contextual information may include information that provides a background or setting where something occurs, allowing for a better understanding of its relevance, implications, or interpretation. Without context, information or actions might be misunderstood or lack significance. Contextual information may include information pertaining to one or more of the following: physical environment (e.g., location, time, surroundings), cultural background (e.g., customs, beliefs, language), social situation (e.g., relationships, roles, social norms), and/or historical or temporal factors (e.g., events leading up to or surrounding the current moment).

In one example, contextual information includes information indicative of and/or contextually relevant to a user's level of knowledge and/or familiarity with respect to a target concept. Information that is indicative of and/or contextually relevant to a user's level of knowledge and/or familiarity with respect to a target concept may include one or more of the following: vocabulary complexity, use of terminology, questions asked, connections made between topics, prior knowledge or experience (evidenced or presumed) with the particular target concept, prior knowledge or experience (evidenced or presumed with a concept that is different from and related to the target concept, types of errors made, types of incorrect statements made, and/or confidence level (evidenced or presumed). Additionally, or alternatively, information that is indicative of and/or contextually relevant to a user's level of knowledge and/or familiarity with respect to a target concept may include information pertaining to one or more of the following: level of education (formal and/or informal), field of study, occupation, field of employment, years of employment, or social networks.

Additionally, or alternatively, contextual information includes a geographic location, such as a user's current location, residential location, or a location the user may visit. Additionally, or alternatively, contextual information may include a time of interaction, such as a time or date when the user interacts with a system or service. Additionally, or alternatively, contextual information may include device usage information, such as use history or the type of devices the user uses (e.g., medical devices, mobile phones, laptops, or other computing devices). Additionally, or alternatively, contextual information may include information pertaining to social connections, such as information pertaining to a user's social network or relationships (e.g., family, friends, or colleagues). Additionally, or alternatively, contextual information may include information pertaining to a user's cultural background, such as information pertaining to language preferences, cultural customs, or traditions. Additionally, or alternatively, contextual information may include information pertaining to a user's interaction history, such as previous interactions with a systems, services, clinicians, or other people. Additionally, or alternatively, contextual information may include information pertaining to a user's behavioral patterns, such as habits, routines, or activity patterns.

In one example, contextual information includes personal data. The term “personal data” refers to data that relates to an identified or identifiable person. As examples, personal data may include data associated with one or more of the following: a name of a person, a nickname, a surname, a maiden name, a familial name, a cultural name, a pseudonym, an alias, a user name, a physical address, a mailing address, an email address, a phone number, a social security number, a driver's license number, a passport number, an ID card number, an insurance ID number, a date of birth, place of birth, gender, race, ethnicity, religion, marital status, familial status, spouse or partner name and information, family member name and information, demographic information, educational background, employment history, income information, financial information, citizenship, or immigration status.

The user device interface 108 is communicatively coupled or couplable with one or more other components of the system 100. The user device interface 108 may include hardware and/or software configured to facilitate interactions between a user and various aspects of the system 100, such as interacting with health information, including explanations of the heath information generated by the content generation engine 104. The user device interface 108 may render user interface elements and receive input via user interface elements. For example, the user device interface 108 may display outputs generated by the system 100. Additionally, or alternatively, the user device interface 108 may be configured to select datasets as inputs to the system 100. Examples of interfaces include a graphical user interface (GUI), a command line interface (CLI), a haptic interface, or a voice command interface. Examples of user interface elements include checkboxes, radio buttons, dropdown lists, list boxes, buttons, toggles, text fields, date and time selectors, command lines, sliders, pages, or forms. Any one or more of these interfaces or interface elements may be utilized by the user device interface 108.

In an embodiment, different components of a user device interface 108 are specified in different languages. The behavior of user interface elements is specified in a dynamic programming language such as JavaScript. The content of user interface elements is specified in a markup language, such as hypertext markup language (HTML) or XML User Interface Language (XUL). The layout of user interface elements is specified in a style sheet language, such as Cascading Style Sheets (CSS). Alternatively, the user device interface 108 may be specified in one or more other languages, such as Java, C, or C++.

The communication interface 110 may be communicatively coupled or couplable with the data repository 102, the content generation engine 104, the ML system 106, and/or the user device interface 108. The communication interface 110 may include hardware and/or software configured to transmit data between respective components of the system 100 and/or to transmit data to and/or from the system 100.

In one or more embodiments, the system 100 may include more or fewer components than the components illustrated in FIG. 1. In one example, the system described with reference to FIG. 1 may include one or more features described below in Section 6, titled “Example Machine Learning System,” Section 7, titled “Computer Networks and Cloud Networks,” and/or Section 8, titled “Hardware Overview.” The components illustrated in FIG. 1 may be local to or remote from each other. The components illustrated in FIG. 1 may be implemented in software and/or hardware. Each component may be distributed over multiple applications and/or machines. Multiple components may be combined into one application and/or machine. Operations described with respect to one component may instead be performed by another component.

In one example, the system 100 may be implemented on one or more digital devices. The term “digital device” generally refers to any hardware device that includes a processor. A digital device may refer to a physical device executing an application or a virtual machine. Examples of digital devices include a computer, a tablet, a laptop, a desktop, a netbook, a server, a web server, a network policy server, a proxy server, a generic machine, a function-specific hardware device, a hardware router, a hardware switch, a hardware firewall, a hardware firewall, a hardware network address translator (NAT), a hardware load balancer, a mainframe, a television, a content receiver, a set-top box, a printer, a mobile handset, a smartphone, a personal digital assistant (PDA), a wireless receiver and/or transmitter, a base station, a communication management device, a router, a switch, a controller, an access point, and/or a browser device.

### 3. EXAMPLE OPERATIONS PERTAINING TO GENERATING EXPLANATIONS OF TARGET CONCEPTS

Referring to FIGS. 2A-2C, example operations pertaining to generating explanations of target concepts are further described. One or more operations 200 described with reference to FIGS. 2A-2C may be modified, combined, rearranged, or omitted. Accordingly, the particular sequence of operations 200 described with reference to FIGS. 2A-2C should not be construed as limiting the scope of one or more embodiments. In one example, the operations 200 may be performed by the one or more components of the system described herein.

### A. Constructing Input Prompt Elements for Generating Knowledge-Adapted Content

Referring to FIG. 2A, the system performs operations 200 pertaining to prompting an ML model to generate a knowledge-adapted content element based on a knowledge classification of a user with respect to a target concept associated with a set of information for display on a user interface. The knowledge classification of the user may include the user's level of knowledge and/or familiarity with the target concept. In one example, the operations pertain to prompting an ML model to generate a knowledge-adapted content element pertaining to a health concept based on a knowledge classification of a patient pertaining to a health concept, such as a level of knowledge and/or familiarity of the patient with respect to the health concept.

As shown in FIG. 2A, the system determines an occurrence of a trigger event corresponding to an interaction with an interface element of a user interface (Operation 202). The trigger event can include a request to display information for a user. Additionally, or alternatively, the trigger event may include an interaction that indicates confusion, such as delay or entering a query that includes inaccurate information. The interface element may be associated with the user. In one example, the interface element is part of a health information interface that allows a patient to access health information based on EHRs of the patient. The trigger event may include a notification generated in response to the interaction with the interface element. The interaction with the interface element may include a user login to the user interface. Additionally, or alternatively, the interaction with the interface element may include accessing a particular section of the user interface and/or accessing information via the user interface. Additionally, or alternatively, the trigger event may include updated information that was added to electronic records associated with the user. The system may determine an occurrence of updated information being added to electronic records associated with the user by monitoring an electronic record system for updates. Additionally, or alternatively, the electronic record system may push notifications of updates to the system, and the system may determine the occurrence of a trigger event in response to receiving a notification of an update.

In response to the trigger event, the system determines whether the trigger event satisfies a trigger condition for requesting a knowledge-adapted content element (Operation 204). When the trigger even does not satisfy the trigger condition, the system continues determining whether additional trigger events that occur at operation 202 satisfy a trigger condition. The knowledge-adapted content element can be utilized to augment a display of a set of information on the user interface based on the knowledge classification of the user with respect to a target concept associated with the set of information for display on the user interface. In one example, the target concepts is a health concept. The health concept may be associated with one or more EHRs pertaining to the user. The trigger condition may include a set of one or more rules, criteria, or conditions that define when the system will generate an explanation of the target concept. In one example, the trigger condition may indicate for the system to generate a knowledge-adapted content element that includes an explanation of a health concept based one or more characteristics of health data associated with the health concept. In one example, the trigger event may satisfy the trigger condition when the health concept corresponds to recent health data, such as health data that was added to an EHRs system within a specified timeframe and/or since a previous time that the user accessed the user interface. For example, the recent health data may include recent health data pertaining to one or more of the following: a recent diagnosis, a recent condition, a recent treatment, a recent visit or appointment, recent clinical notes, a recent surgery, a recent radiology image, recent test results, or recently prescribed medications. In one example, the trigger event may satisfy the trigger condition when the target concept is related to one or more other target concepts.

When the trigger event satisfies the trigger condition for requesting the knowledge-adapted content element, the system generates, in real time, an input prompt element for requesting the knowledge-adapted content element (Operation 206). The input prompt element includes an instruction for an ML model to generate the knowledge-adapted content element. The input prompt element may instruct the ML model to generate the knowledge-adapted content element based on a knowledge classification of the user with respect to the target concept. In one example, the input prompt element instructs the ML model to generate a knowledge-adapted content element that includes an explanation of a target concept. The explanation of the target concept is based on a knowledge classification of the user. The knowledge classification of the user may include a depth of knowledge of the user and/or a familiarity of the user with respect to the target concept. The knowledge classification of the user can be stored in a profile, submitted in the input prompt to the ML model, or generated by the ML model based on contextual information. In one example, the input prompt element may instruct the ML model to generate the knowledge classification of the user with respect to the target concept, for example, based on a predicted depth of knowledge of the user and/or a familiarity of the user with respect to the target concept. Additionally, or alternatively, the input prompt element may instruct the ML model to generate a prediction of the depth of knowledge of the user and/or the familiarity of the user with respect to the target concept and then generate the knowledge-adapted content element based on the prediction. The knowledge classification of the user may include the prediction.

The system may construct the input prompt element automatically in response to determining that the trigger event satisfies the trigger condition. The input prompt element may include information pertaining to the user, the trigger event, and/or the trigger condition. In one example, the input prompt element includes the target concept. Additionally, or alternatively, the input prompt element may include target concept data associated with the user and/or information that enables the ML model to access an electronic records system and retrieve target concept data associated with the user. In one example, the target concept is a health concept. In one example, the input prompt element includes a patient identification code for accessing health data from EHRs associated with the patient. Additionally, or alternatively, the input prompt element may include contextual data and/or information that enables the ML model to access contextual data from a data repository. The contextual data and the target concept data may be located in the same data repository or different data repositories. The system may access contextual data and/or target concept data from multiple data repositories.

After constructing the input prompt element, the system directs the input prompt element to the ML model (Operation 208). In one example, the system utilizes an application programming interface (API) to direct the input prompt element to the ML model. Additionally, or alternatively, the system may utilize a CLI to direct the input prompt element to the ML model. A software application may enter the input prompt element as a command-line argument. The CLI may include a CLI script that directs the input prompt element to the ML model. Additionally, or alternatively, a software application may include instructions for constructing input prompt elements and directing the input prompt elements to the ML model. In one example, the system includes an automated data pipeline where input prompt elements are constructed and/or received from one or more sources and automatically directed to the ML model.

Based at least in part on the input prompt element, the ML model executes an inference that includes generating the knowledge-adapted content element. The ML model may generate the knowledge-adapted content element based on the knowledge classification of the user with respect to the target concept. The system receives, from the ML model, an output that includes knowledge-adapted content element (Operation 210). The knowledge-adapted content element includes machine-generated content pertaining to the target concept. In one example, the system receives the output from the ML model in a structured format, such as JSON, XML, or plain text. The system may parse the output to extract the knowledge-adapted content element and/or machine-generated content pertaining to the target concept included in the knowledge-adapted content element. For example, if the output is a JSON object that includes multiple fields, the system may identify a field that includes the machine-generated content pertaining to the target concept. The system may apply data formatting to the output. Additionally, or alternatively, the system may apply labels or tags to the knowledge-adapted content element that allows the system to incorporate the machine-generated content with other information displayed by the user interface.

After receiving the output that includes the knowledge-adapted content element and/or after extracting the machine-generated content pertaining to the target concept, the system stores and/or transmit at least a portion of the knowledge-adapted content element (Operation 212). The system may store and/or transmit at least the portion of the knowledge-adapted content element to enable the system to augment a set of information for display on a user interface based on the knowledge-adapted content element. In one example, the system may store and/or transmit the machine-generated content pertaining to the target concept. In one example, the system may display the machine-generated content from the knowledge-adapted content element on the user interface concurrently with displaying the set of information on the user interface. In one example, the system transmits a message to the user interface that includes the knowledge-adapted content element and/or the machine-generated content pertaining to the target concept. Additionally, or alternatively, the system may store the knowledge-adapted content element and/or the machine-generated content pertaining to the target concept in a data repository for retrieval by the system. The system may retrieve the knowledge-adapted content element and/or the machine-generated content pertaining to the target concept based on a tag or label applied to the knowledge-adapted content element and/or the machine-generated content.

The system may evaluate multiple trigger events against one or more trigger conditions for generating knowledge-adapted content elements. As shown in FIG. 2A, the system determines whether an additional trigger event has occurred (Operation 214). When an additional trigger event has occurred, the system determines whether the trigger event satisfies a trigger condition for generating knowledge-adapted content elements as described above.

### B. Executing ML Inferences to Generate Knowledge-Adapted Content Elements

Referring to FIG. 2B, the system performs operations 220 pertaining to executing ML inferences to generate knowledge-adapted content elements. As shown in FIG. 2B, an ML model receives an input prompt element that includes an instruction for an ML model to generate a knowledge-adapted content element based on a knowledge classification of a user with respect to a target concept (Operation 222). The target concept may be located in a set of one or more electronic records of the patient. The input prompt element may be constructed as described above with reference to FIG. 2A. The input prompt element may be directed to the ML model, and/or the ML model may retrieve the input prompt element from a data repository. The ML model determines the target concept based on the input prompt element and/or the target concept dataset. In one example, the input prompt element indicates that a target concept is available in a target concept dataset. For example, the input prompt element may indicate that a health concept is available in one or more EHRs located in an EHRs system. In one example, the ML model identifies the target concept based on a timestamp.

Based on the input prompt element, the ML model accesses a target concept dataset (Operation 224). In one example, the target concept dataset includes a set of one or more EHRs of a patient. The set of one or more EHRs may include the target concept. In one example, the target concept is a health concept. The target concept and/or the target concept dataset may be provided with the input prompt element. Additionally, or alternatively, the ML model may access the target concept and/or the target concept dataset from an electronic records system. The input prompt element may include information that enables the ML model to access the target concept and/or the target concept dataset from the electronic records system.

In one example, based on the input prompt element, the ML model accesses a contextual information dataset that includes contextual information pertaining to the user (Operation 226). In one example, the ML model accesses the contextual information dataset from a data repository. The input prompt element may include information that enables the ML model to access the contextual information dataset from the data repository. Additionally, or alternatively, the input prompt element may include the contextual information dataset.

In one example, after accessing the target concept dataset and the contextual information dataset, the ML model executes an inference to determine, based on the target concept dataset and/or the contextual information dataset, a set of predictive information that is relevant for determining a knowledge classification of the user with respect to the target concept (Operation 228). In one example, the predictive information is relevant for predicting a level of knowledge and/or familiarity of the user with the target concept. Additionally, or alternatively, the ML model may access the set of predictive information based on the input prompt element.

After determining and/or accessing the set of predictive information, the ML model executes an inference to determine the knowledge classification of the user with respect to the target concept (Operation 230). The knowledge classification of the user may include a predicted level of knowledge and/or familiarity of the user with respect to the target concept. The ML model may generate a natural language explanation of a predicted user comprehension of the target concept. The natural language explanation may include a description of multiple types of knowledge pertaining to the target concept. Additionally, or alternatively, the natural language explanation may address multiple aspects of the target concept.

The ML model may generate a predicted user comprehension of the target concept based at least in part on the target concept and a set of contextual user information associated with the user. The ML model may determine different knowledge classifications for a particular user with respect to different target concepts and/or with respect to different aspects of a particular target concept. The different knowledge classifications may reflect different levels and/or depths of knowledge or familiarity with respect to different target concepts and/or different aspects of a particular target concept. In one example, the knowledge classification of the user with the target concept includes an identification of one or more features of the target concept that the ML model predicts that the user is likely to understand and/or that the user is likely to be familiar with. Additionally, or alternatively, the knowledge classification of the user may include an identification of one or more features of the target concept that the ML model predicts that the user is unlikely to understand and/or that the user is likely unfamiliar with. For example, the ML model may identify a set of features of the target concept and categorize the features of the target concepts based on the knowledge classification of the user.

In one example, the knowledge classification of the user includes a prediction as to one or more of the following types of knowledge: factual knowledge, conceptual knowledge, procedural knowledge, metacognitive knowledge, declarative knowledge, or strategic knowledge. Factual knowledge includes knowledge about facts. Conceptual knowledge includes knowledge about relationships between elements or concepts within a larger structure or system. Procedural knowledge includes knowledge about methods, techniques, or processes for accomplishing objectives. Metacognitive knowledge of a person includes the person's ability to recognize when they do or do not understand a concept and apply strategies to address gaps in understanding. Declarative knowledge includes the ability to communicate knowledge. Strategic knowledge includes knowing how to prioritize tasks, decision-making knowledge, and problem-solving skills.

In one example, the knowledge classification of the user includes a predicted depth of knowledge. The predicted depth of knowledge may include one or more of the following: surface knowledge, basic understanding, deeper understanding, application knowledge, critical knowledge, or synthesis knowledge. Surface knowledge includes a basic recognition or recall of facts without deeper understanding of mechanisms or principles. Basic understanding includes simple connections between facts or ideas, for example, with some understanding of cause-and-effect relationships. Deeper understanding includes knowledge about underlying processes, relationships, and principles that explain why or how something works. Application knowledge includes being able to use knowledge to solve problems, make decisions, or implement knowledge in real-world or novel contexts. Critical knowledge includes an ability to analyze, evaluate, and make judgements based on criteria, evidence, and understanding of broader implications. Synthesis knowledge includes an ability to combine information from various sources to create new ideas, approaches, or frameworks.

In one example, for a health concept pertaining to a new diagnosis, a patient that is unfamiliar with the health concept may have surface level knowledge about the health concept after an appointment with a physician where the diagnosis is introduced to the patient. A patient that has received treatment for the diagnosis for a period of time may have increased familiarity with the diagnosis based on the treatment received. The patient may have a basic understanding of a health concept pertaining to the diagnosis, for example, based on experiences and/or conversations with clinicians pertaining to those treatments. A patient that has formal education or training in a health-related field may have a deeper understanding of a health concept. In one example, even with formal education or training, a patient may have a lower depth of knowledge with a health concept that is new or unconventional.

In one example, the system determines the knowledge classification of the user based on a query submitted by the user. Based on the query, the system may determine a comprehension score. The system may map the comprehension score to a target concept. The system may determine that the comprehension score satisfies a comprehension threshold that represents a level of comprehension of the user with respect to the target concept. Additionally, or alternatively, the system may determine the knowledge classification of the user based on a complexity of a target concept. The system determines a complexity score for the target concept. The complexity score represents a level of complexity of the target concept. The system may map the complexity score to the target concept. The system may determine the knowledge classification of the user based on a combination of the comprehension score and the complexity score.

In one example, the system determines the knowledge classification of the user based on a record date. The record date may represent a date that an electronic record was created or made available to the user. The system may determine that the record date satisfies a date threshold. The date threshold may represent a time period for the user to gain familiarity with the target concept. The system may infer a level of comprehension of the user based on the time period that the user has had access to information in the electronic record. In one example, relatively new information satisfies a trigger condition based on the user presumably being less familiar with relatively newer information. Additionally, or alternatively, relatively older information may not satisfy the trigger condition based on the user presumably being more familiar with relatively older information. Additionally, or alternatively, the system may determine the knowledge classification of the user based on an encounter score. The encounter score is based on one or more previous interactions with a user interface pertaining to the target concept. The encounter score may represent one or more aspects of the one or more previous interactions, such as depth of content consumption, repeat interactions, or cognitive effort signals, such as time spent interacting, queries submitted, or types of interactions.

In one example, the knowledge classification of the user includes one or more example explanations of example topics adapted to the knowledge classification of the user. The system may determine the example explanations based on a predicted user comprehension of the target concept. The system may generate the knowledge-adapted content based on the example explanations, as described below.

Based on the knowledge classification of the user with respect to the target concept, the system executes an inference to generate an output that includes the knowledge-adapted content element (Operation 232). The ML model may generate a natural language explanation of the target concept in context with other information for display on the user interface based on a predicted user comprehension of the target concept. The ML model may utilize a natural language explanation of the predicted user comprehension as an input for generating the natural language explanation of the target concept.

The ML model may generate the explanation of the target concept based on grounding information corresponding to the knowledge classification of the user. The system may generate the grounding information and utilize the grounding information as an input for generating the explanation of the target concept. The grounding information may include at least one of the following: a vocabulary set associated with the target concept for use generating machine-generated content, a length guideline for a length of the machine-generated content, or a formatting guideline for formatting the machine-generated content. The input to the ML model may include the target concept and the grounding information. The ML model may generate the explanation of the target concept adapted to the knowledge classification of the user based at least in part on the grounding information.

In one example, the ML model determines a topic that would be helpful for the user to understand, then the ML model explains the topic based on an example explanation adapted to the knowledge-classification of the user. The topic may include a health topic, for example, based on a diagnosis of the user. The system may access a set of health information in an EHR associated with a diagnosis of the user and determine the target concept based at least in part on the diagnosis of the user. The target concept may include a topic pertaining to improving user understanding of the diagnosis. The ML model may generate machine-generated content pertaining to the target concept based on an example explanation of an example topic adapted to the knowledge classification of the user.

The ML model may generate the knowledge-adapted content element based on one or more inferences. For example, operations 228-232 may represent elements of a combined inference and/or separate inferences. Additionally, operations 228-232 may represent inferences performed utilizing separate ML algorithms or a same ML algorithm. The ML model may include and/or exclude machine-generated content from the knowledge-adapted content element based on the knowledge classification of the user. Additionally, or alternatively, the ML model may determine a communication style based on the knowledge classification of the user and may generate machine-generated content for the knowledge-adapted content element utilizing the communication style.

The ML model may generate knowledge-adapted content elements for multiple target concepts. As shown in FIG. 2B, the ML model determines whether there is an additional input prompt element that includes an instruction to generate an additional knowledge-adapted content element for an additional target concept (Operation 234). When the ML model determines that there is an additional input prompt element that includes an instruction to generate an additional knowledge-adapted content element for an additional target concept, the ML model generates the additional knowledge-adapted content element for the additional target concept. As shown in FIG. 2B, to generate the additional knowledge-adapted content element, the system returns to operation 224, to access a target concept dataset for generating the additional knowledge-adapted content element. When the system determines that there is not an additional input prompt that includes an instruction to generate an additional knowledge-adapted content element at operation 234, the system waits for additional input prompts, and when an additional input prompt is received, the system determines whether the input prompt includes an instruction to generate an additional knowledge-adapted content element.

### C. Displaying Knowledge-Adapted Content in Response to Interactions with Interface Elements

Referring to FIG. 2C, the system performs operations 240 pertaining to displaying machine-generated content from knowledge-adapted content elements on user interfaces. The system may render machine-generated content from a knowledge-adapted content element in response to an interaction with an interface element of a user interface. As shown in FIG. 2C, the system determines an interaction by a user with an interface element of a user interface (Operation 242). The interaction with the interface element may include one or more of the following: accessing the user interface, opening a page of the user interface, clicking on the interface element, selecting the interface element, selecting an option on the user interface, or entering a query on the user interface. The interface element may include and/or may be associated with a display of a set of information associated with the target concept on the user interface. Additionally, or alternatively, the target concept may be displayed on the user interface in connection with machine-generated content from the knowledge-adapted content element when the system generates the knowledge-adapted content element.

In response to the interaction with the interface element, the system generates a trigger event (Operation 244). The system utilizes the trigger event for determining whether to generate a knowledge-adapted content element that includes machine-generated content for display on the user interface. The machine-generated content is generated based on a knowledge-classification of the user with respect to a target concept. The target concept may be located in a set of EHRs pertaining to the patient. The trigger event may include an identification of the interaction with the interface element and/or an identification of the target concept associated with the interface element.

The system directs the trigger event to a content generation engine (Operation 246). The content generation engine determines the occurrence of the trigger event. Additionally, or alternatively, the content generation engine determines whether the trigger event satisfies a trigger condition for generating a knowledge-adapted content element. Operations pertaining to determining the occurrence of the trigger event and determining whether the trigger event satisfies the trigger condition are described above with reference to FIG. 2A.

When the trigger event satisfies the trigger condition and the system generates a knowledge-adapted content element, the system receives an output from the content generation engine that includes the knowledge-adapted content element (Operation 248). In response to receiving the output, the system augments a set of information for display on the user interface based on the knowledge-adapted content element (Operation 250). In one example, the system augments the set of information by displaying machine-generated content from the knowledge-adapted content element on the user interface concurrently with displaying the set of information.

The system may augment information pertaining to multiple target concepts based on knowledge-adapted content elements in response to multiple trigger conditions associated with interactions with various interface elements of the user interface. As shown in FIG. 2C, the system determines whether there is an additional interaction with an interface element of the user interface (Operation 252). When the system determines that there is an additional interaction with an interface element of the user interface, the system returns to operation 244 and generates an additional trigger event in response to the additional interaction and directs the trigger event to the content generation engine as described above. If the trigger event satisfies a trigger condition, the system receives an additional knowledge-adapted content element corresponding to the additional interaction with the interface element. When the system determines that there is not an additional interaction with an interface element of the user interface at operation 252, the system waits for additional interaction with an interface element of the user interface, and when an additional interaction with an interface element of the user interface is detected, the system returns to operation 244 and generates an additional trigger event in response to the additional interaction.

In one example, in response to an interaction with an interface element for displaying health information, the system identifies a target concept in the health information and generates a natural language explanation of the target concept. Next, the system generates knowledge-adapted health information by combining the explanation with the health information. The system detects an interaction with an interface element of the user interface. In response to detecting the interaction with the interface element, the system identifies a mapping between the interface element and the EHR that includes a set of health information associated with the user. The system accesses the set of health information in an electronic records repository. The system directs a model input, including the set of health information, to the ML model. The ML model is trained to identify particular concepts based on sets of health information that, when explained according to different knowledge classifications of different users, at least partially contribute to improving user understanding. The system receives a response from the ML model. The response includes a target concept determined by the ML model based on the input. The trigger condition for generating the knowledge-adapted content element includes receiving the response. The ML model generates the knowledge-adapted content element based on the target concept identified by the model. The knowledge-adapted content element may include an explanation of the target concept adapted to the knowledge classification of the user. The system may combine the explanation of the target concept with at least a portion of the set of health information. The system may augment the set of information by displaying the explanation of the target concept with at least the portion of the set of health information.

In one example, in response to an interaction with a visit summary interface for displaying visit summaries for healthcare services provided to a user, the system generates a visit summary by combining health information pertaining to the healthcare services with a knowledge-adapted explanation of the health information. The system detects an interaction with the visit summary interface. In response to detecting the interaction with the visit summary interface, the system accesses an electronic records repository and extracts a set of health information from one or more EHRs of the user stored in the electronic records repository. The system directs a model input, including the set of health information, to an ML model. The ML model is trained to identify particular concepts based on sets of health information that, when explained according to different knowledge classifications of different users, at least partially contribute to improving user understanding. The system receives a response from the ML model. The response includes a target concept determined by the ML model based on the input. The trigger condition for generating the knowledge-adapted content element includes receiving the response. The ML model generates the knowledge-adapted content element based on the target concept identified by the model. The knowledge-adapted content element may include an explanation of the target concept adapted to the knowledge classification of the user. The system generates a visit summary based on the knowledge-adapted content element. The system may generate the visit summary by combining the explanation of the target concept with at least a portion of the set of health information. The system may augment the set of health information by displaying the visit summary on the visit summary interface.

In one example, the system receives a query via the user interface. In response to receiving the query, the system determines a target concept based on the query, generates a natural language explanation of the target concept, then generates a knowledge-adapted query response by combining the explanation with a query response generated in response to the query. The system detects an interaction with an interface element of the user interface. The interaction includes receiving a query. The system directs an input to an ML model that includes the query. The ML model is trained to identify target concepts based on queries that, when explained according to different knowledge classifications of different users, at least partially contribute to improving user understanding. The system receives a response from the ML model. The response includes a target concept determined by the ML model based on the input. The trigger condition for generating the knowledge-adapted content element includes receiving the response. The system generates a query response in response to the query. The ML model generates the knowledge-adapted content element based on the target concept identified by the model. The knowledge-adapted content element may include an explanation of the target concept adapted to the knowledge classification of the user. The system may combine the explanation of the target concept with at least a portion of the query response. The system may augment the set of information by displaying the explanation of the target concept with at least the portion of the query response.

In one example, the system receives a query response. The query response can be generated in response to an interaction with the user interface such as submitting a query. In response to receiving the query response, the system determines a target concept based on the query response, generates a natural language explanation of the target concept, then generates a knowledge-adapted query response by combining the explanation with the query response. The system receives a query response. The system directs an input to an ML model that includes the query response. The ML model is trained to identify target concepts based on query responses that, when explained according to different knowledge classifications of different users, at least partially contribute to improving user understanding. The system receives a response from the ML model. The response includes a target concept determined by the ML model based on the input. The trigger condition for generating the knowledge-adapted content element includes receiving the response. The ML model generates the knowledge-adapted content element based on the target concept identified by the model. The knowledge-adapted content element may include an explanation of the target concept adapted to the knowledge classification of the user. The system may combine the explanation of the target concept with at least a portion of the query response. The system may augment the set of information by displaying the explanation of the target concept with at least the portion of the query response.

### 4. EXAMPLE USER INTERFACES

A detailed example is described below for purposes of clarity. Components and/or operations described below should be understood as specific examples that may not be applicable to certain embodiments. Accordingly, components and/or operations described below should not be construed as limiting the scope of any of the claims.

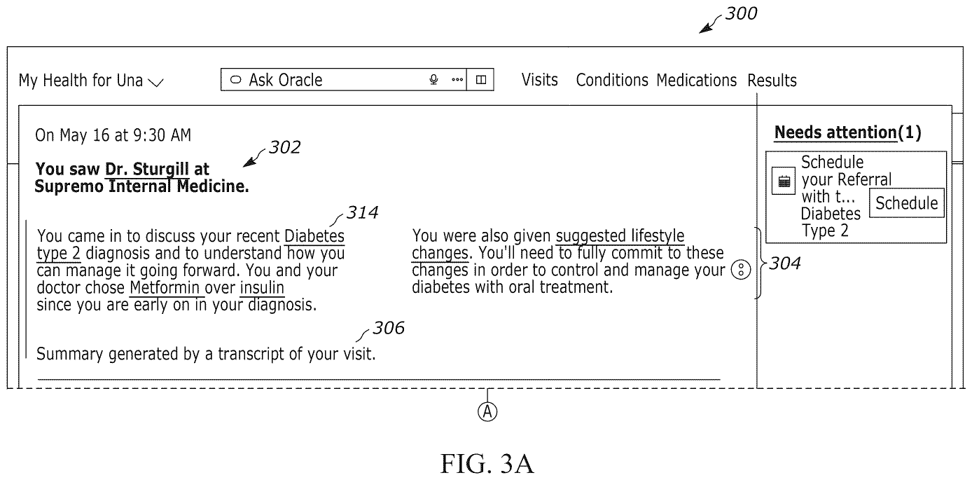

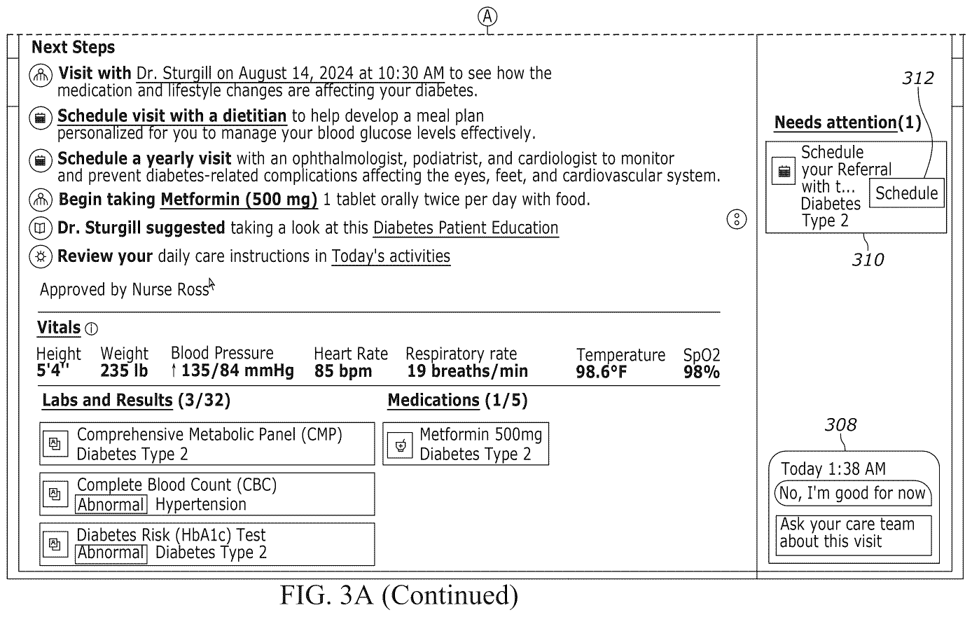

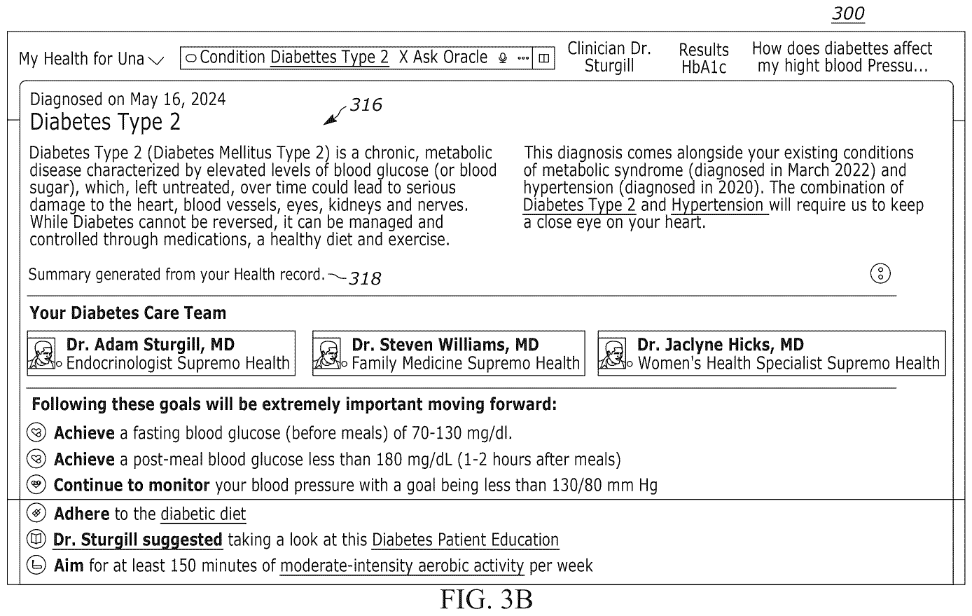

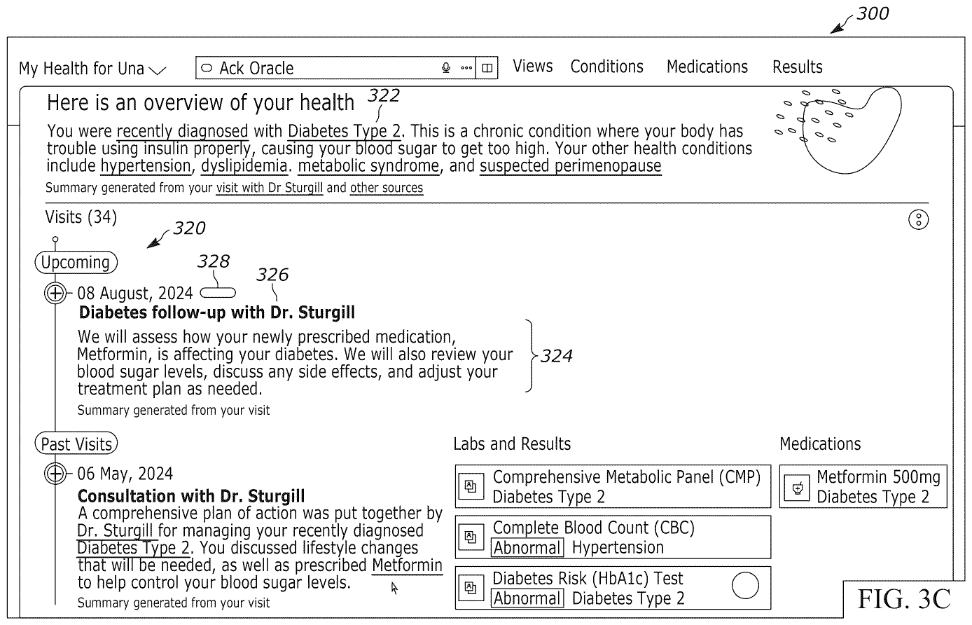

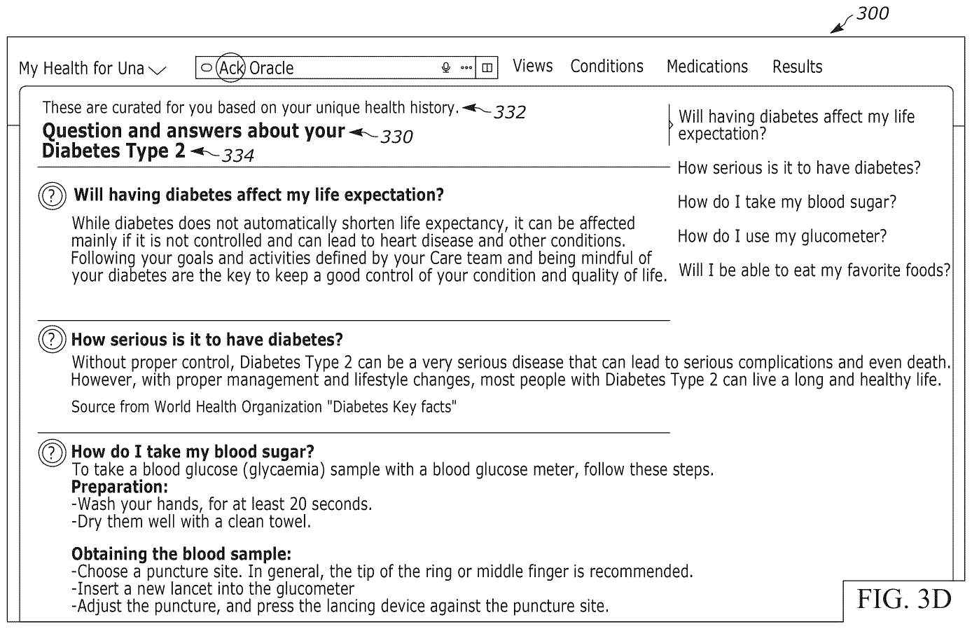

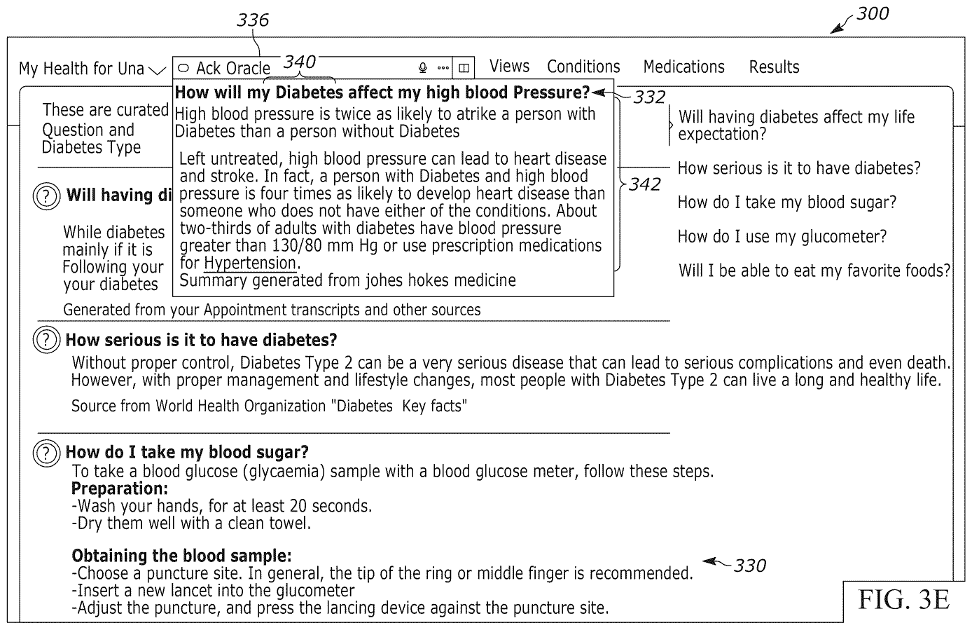

FIGS. 3A-3E show examples of a user interface 300 that includes information that is augmented based on machine-generated content from knowledge-adapted content elements pertaining to various target concepts. As shown in FIGS. 3A-3E, the user interface 300 may include a health information interface. Additionally, or alternatively, as shown in FIGS. 3A-3E, the various target concepts may include health concepts.

As shown in FIG. 3A, a user interface 300 includes a patient summary called “My Health.” The patient summary includes a visit summary 302. The visit summary includes machine-generated content 304 from a knowledge-adapted content element. The knowledge-adapted content element is generated based on a knowledge-classification of the user with respect to a target concept such as a health concept. The system may generate the machine-generated content 304 in response to the patient accessing the patient summary. The machine-generated content 304 may include and/or exclude content as determined by the ML model based on the knowledge classification of the user. Additionally, or alternatively, the machine-generated content 304 may utilize a communication style determined by the ML model based on the knowledge classification of the user. The knowledge classification of the user may be determined based on contextual information.

In one example, as shown in FIG. 3A, the target concept is a health concept, such as a diagnosis and/or a concept pertaining to the diagnosis. Additionally, or alternatively, as shown in FIG. 3A, the target concept may pertain to a visit with a clinician. In one example, the contextual information utilized for determining the knowledge classification of the user may include visit information 306, such as clinician nodes and/or a transcript of the visit. In one example, the information that is augmented by the system based on the knowledge-adapted content element includes the visit information 306. The system generates the visit summary 302 based on the contextual information associated with the visit. The machine-generated content 304 includes a summary of what was discussed in the appointment. Additionally, or alternatively, the machine-generated content 304 may include next steps the patient needs to take that have been approved by someone in the care team. Additionally, or alternatively, the machine-generated content 304 may include labs, results, and/or medications that were prescribed or reviewed in connection with the visit. In one example, the system utilizes a communication style in the machine-generated content 304 based on the knowledge classification of the patient.

In another example, the information that is augmented by the system based on the knowledge-adapted content element may include a chat dialogue 308. As shown in FIG. 3A, the chat dialogue 308 indicates that the patient should ask their care team about the visit. The system generates the visit summary 302 based on the context of this chat dialogue 308. The chat dialogue 308 may have occurred prior to and/or when scheduling the visit with the clinician. The system includes the chat dialogue 308 as part of the knowledge-adapted content element pertaining to the visit summary because the ML model has determined that the chat dialogue 308 is contextually relevant to the visit.

In another example, the machine-generated content 304 may include an action item 310 for the patient to perform. The action item 310 may include an interface element 312 for commencing the action item 310. As shown in FIG. 3A, the action item 310 includes scheduling an appointment. The interface element 312 includes a button to schedule the appointment.

In another example, when the patient clicks on an interface element 314, the patient is taken to a next page of the user interface 300 shown in FIG. 3B. The interface element 314 may include details associated with the visit summary. For example, as shown in FIG. 3B, the interface element 314 includes a diagnosis associated with the visit summary.

As shown in FIG. 3B, the system generates machine-generated content 316 of the details, such as the diagnosis, associated with the visit summary. The system may generate the machine-generated content 316 based on target concept data 318, such as an EHR of the patient. The machine-generated content 316 may include and/or exclude content as determined by the ML model based on the knowledge classification of the user. Additionally, or alternatively, the machine-generated content 316 may utilize a communication style determined by the ML model based on the knowledge classification of the user. In one example, the knowledge classification of the user is based at least in part on a timeframe associated with the diagnosis. The system generates the machine-generated content 316 based on the timeframe. The system may update the machine-generated content 316 as the timeframe changes. As shown, the machine-generated content 316 is based on a timeframe corresponding to a new diagnosis. As shown, the machine-generated content 316 includes information about the diagnosis that is introductory in nature. The system may determine different timeframes based on target concept data, such as EHRs of the patient. As the timeframe changes, the system may change the machine-generated content 316 and/or the communication style of the machine-generated content 316. For example, after the patient has been living with the diagnosis for a while, the machine-generated content 316 may include information about how to improve day-to-day living. In another example, when the EHRs of the patient include an additional diagnosis, the machine-generated content 316 may include information pertaining to how the additional diagnosis relates to and/or impacts the initial diagnosis. For example, as shown in FIG. 3B, the machine-generated content 316 describes a relationship between diabetes type 2 and hypertension. As another example, if a patent enters into a pregnancy, the machine-generated content 316 may include information that describes how the pregnancy impacts the diagnosis and/or how management of the diagnosis may change as a result of the pregnancy. In another example, the machine-generated content 316 may include educational information. The system may update the machine-generated content 316 with different or additional educational information as the patient progresses through an educational process. The knowledge classification of the user can be based at least in part on the user's previous interactions with the user interface 300.

Referring to FIG. 3C, in one example, the user interface 300 incudes a set of visits 320 associated with a particular diagnosis 322. The set of visits 320 may include current and/or past visits. The system may filter the set of visits 320 based on the particular diagnosis 322. For example, the system may include visits that are contextually relevant to the particular diagnosis 322, and/or the system may exclude visits that are not contextually relevant. The system may generate machine-generated content 324 associated with one or more visits in the set of visits 320 such as an upcoming visit 326. Additionally, or alternatively, as shown in FIG. 3C, the machine-generated content 324 may include labs, results, and/or medications associated with the particular diagnosis 322.

The system may generate the machine-generated content 324 based on target concept data 328 such as a schedule entry in an EHR of the patient. The machine-generated content 324 may include and/or exclude content as determined by an ML model based on the knowledge classification of the user. Additionally, or alternatively, the machine-generated content 324 may utilize a communication style determined by the ML model based on the knowledge classification of the user.

Referring to FIG. 3D, the user interface 300 may include a set of questions and answers 330. The system may generate the set of questions and answers 330 based on one or more target concepts 332 located in an EHR of the patient. For example, the system may generate questions and answers 330 that are contextually relevant to a target concept 332. As shown in FIG. 3D, the system determines that the patient has a diagnosis 334 and generates machine-generated questions and answers 330 that are contextually relevant to the diagnosis 334. The system may select the questions and/or the answers from a data repository based on the knowledge classification of the user. Additionally, or alternatively, the system may generate the questions and/or the answers based on the knowledge classification of the user. In one example, the system may generate the questions and/or the answers based on information provided to the patient during a visit. For example, the system may determine information that is beneficial for the patient to know regarding content in the patients EHRs and formulate an explanation of that information in the form of questions and answers 330 that are adapted to the knowledge classification of the user. The machine-generated content 324 may include and/or exclude questions and answers 330 as determined by an ML model based on the knowledge classification of the user. Additionally, or alternatively, the questions and answers 330 may utilize a communication style determined by the ML model based on the knowledge classification of the user.

Referring to FIG. 3E, in one example, the user interface 300 includes an interface element 336 that allows a patient to input a query 338. For example, as shown in FIG. 3E, the query 338 may include a question about a target concept 340, such as a diagnosis and/or a concept pertaining to the diagnosis. In one example, as shown in FIG. 3E, the query 338 may include a question from the patient about how one diagnosis (e.g., diabetes) impacts another diagnosis (e.g., high blood pressure). The system generates machine-generated content 342 that includes a response to the query 338 adapted to the knowledge classification of the user. For example, as shown in FIG. 3E, the machine-generated content 342 includes an answer to the question from the patient. The system may include and/or exclude content from the answer as determined by an ML model based on the knowledge classification of the user. Additionally, or alternatively, the system may utilize a communication style determined by the ML model based on the knowledge classification of the user. In one example, the system may determine and/or update a set of questions and answers 330 based on the query 338. For example, based on one question asked by the patient in the query 338, the system may determine and/or update the set of questions and answers 330 to include likely follow-up questions to the question asked in the query 338 as well as answers to those follow-up questions.

### 5. PRACTICAL APPLICATIONS, ADVANTAGES, AND IMPROVEMENTS

Embodiments provide several practical applications, advantages, and improvements. By generating knowledge-adapted content, such as explanations of target concepts, in response to trigger events that satisfy trigger conditions, the system is able to timely provide content tailored to the level of knowledge and/or familiarity of the user for display on the user interface. Additionally, the system improves the utilization of resources associated with the ML model by limiting the generation of knowledge-adapted content to scenarios where the particular trigger conditions are satisfied. Additionally, the knowledge-adapted content is generated for particular target concepts, for example, rather than entire sets of information. As a result, resource are utilized to generate the knowledge-adapted content for the particular target concepts corresponding to a satisfied trigger condition. Moreover, the system can generate the knowledge-adapted content for different target concepts based on different knowledge classifications. As a result, different portions of information displayed on the user interface are adapted to the different knowledge classifications. Furthermore, when the trigger events are based on interactions with the user interface, the explanations are provided in an on-demand manner when the user accesses the information. Thus, the resources associated with the ML model are utilized to generate knowledge-adapted content for target concepts relevant to potions of the user interface that are being accessed by the user. In one example, the trigger condition can include a user interaction that is indicative of possible user confusion. By generating knowledge-adapted content in response to a trigger event that satisfies a trigger condition indicative of possible user confusion, the system proactively responds to the possibility of user confusion. The proactive response based on the trigger condition improves system efficiency in providing information to users and reduces demand on system resources for responding to queries pertaining to target concepts that are confusing to the user.

In one example, the system generates a knowledge classification that includes a natural language explanation of a predicted user comprehension of the target concept. The system may utilize the natural language explanation of the predicted user comprehension as an input to generate knowledge-adapted content that includes a natural language explanation of the target concept. ML models that are built around the structure of language exhibit improved computational performance when executing inferences on inputs based on natural language. For example, ML models process sequences of tokens and infer relationships between them. Natural language represents a rich source of information for the ML model to execute inferences. Natural language is open-ended, relational, and compositional. Additionally, natural language carries context, including implicit structures, constraints, exceptions, and dependencies. By contrast, structured inputs, such as flags or database encodings, represent a more limited source of information. By utilizing a knowledge classification that includes a natural language explanation of a predicted user comprehension of the target concept, the ML model is better able to generate knowledge-adapted content tailored to the particular aspects of the user's comprehension with respect to the target concept.

The data input to any ML model and/or the data output from any ML model, as described herein, may be used for operations performed by one or more of the following software: Database Software, Cloud Infrastructure Software, Customer Relationship Management Software, Data Science Software, Digital Assistant Software, Vision Software, Language Software, Speech Software, Forecasting Software, Enterprise Software, Middleware, Server Software, Identity Management Software, Application Development Software, Analytics Software, Security Software, Data Integration Software, Health Software, Hospitality Software, Retail Software, Utilities Software, Operating Systems, Virtualization Software, Governance and Administration Software, Migration & Disaster Recovery Software, Networking Software, Connectivity Software, Monitoring Software, Procurement Software, Project Management Software, Risk Management Software, Supply Chain Management Software, Manufacturing Software, Human Capital Management Software, Customer Experience Software, Advertising Software, and Industry-Specific Application Software.

### 6. EXAMPLE MACHINE LEARNING SYSTEM

### A. Example Architecture of a Machine Learning System

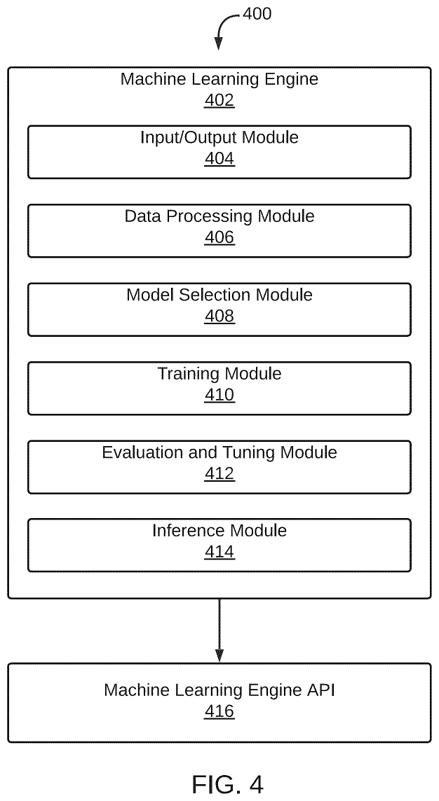

FIG. 4 illustrates an example architecture of an ML system 400. The ML system 400 includes an ML engine 402 in accordance with one or more embodiments. As illustrated in FIG. 4, ML engine 402 includes input/output module 404, data preprocessing module 406, model selection module 408, training module 410, evaluation and tuning module 412, and inference module 414.

In accordance with an embodiment, input/output module 404 serves as the primary interface for data entering and exiting the system, managing the flow and integrity of data. This module may accommodate a wide range of data sources and formats to facilitate integration and communication within the ML architecture.

In an embodiment, an input handler within input/output module 404 includes a data ingestion framework capable of interfacing with various data sources, such as databases, APIs, file systems, and real-time data streams. This framework is equipped with functionalities to handle different data formats (e.g., CSV, JSON, XML) and efficiently manage large volumes of data. It includes mechanisms for batch and real-time data processing that enable the input/output module 404 to be versatile in different operational contexts, whether processing historical datasets or streaming data.

In accordance with an embodiment, input/output module 404 manages data integrity and quality as it enters the system by incorporating initial checks and validations. These checks and validations ensure that incoming data meets predefined quality standards, like checking for missing values, ensuring consistency in data formats, and verifying data ranges and types. This proactive approach to data quality minimizes potential errors and inconsistencies in later stages of the ML process.

In an embodiment, an output handler within input/output module 404 includes an output framework designed to handle the distribution and exportation of outputs, predictions, or insights. Using the output framework, input/output module 404 formats these outputs into user-friendly and accessible formats, such as reports, visualizations, or data files compatible with other systems. Input/output module 404 also ensures secure and efficient transmission of these outputs to end-users or other systems in an embodiment and may employ encryption and secure data transfer protocols to maintain data confidentiality.

In accordance with an embodiment, data preprocessing module 406 transforms data into a format suitable for use by other modules in ML engine 402. For example, data preprocessing module 406 may transform raw data into a normalized or standardized format suitable for training ML models and for processing new data inputs for inference. In an embodiment, data preprocessing module 406 acts as a bridge between the raw data sources and the analytical capabilities of ML engine 402.

In an embodiment, data preprocessing module 406 begins by implementing a series of preprocessing steps to clean, normalize, and/or standardize the data. This involves handling a variety of anomalies, such as managing unexpected data elements, recognizing inconsistencies, or dealing with missing values. Some of these anomalies can be addressed through methods like imputation or removal of incomplete records, depending on the nature and volume of the missing data. Data preprocessing module 406 may be configured to handle anomalies in different ways depending on context. Data preprocessing module 406 also handles the normalization of numerical data in preparation for use with models sensitive to the scale of the data, like neural networks and distance-based algorithms. Normalization techniques, such as min-max scaling or z-score standardization, may be applied to bring numerical features to a common scale, enhancing the model's ability to learn effectively.

In an embodiment, data preprocessing module 406 includes a feature encoding framework that ensures categorical variables are transformed into a format that can be easily interpreted by ML algorithms. Techniques like one-hot encoding or label encoding may be employed to convert categorical data into numerical values, making them suitable for analysis. The module may also include feature selection mechanisms, where redundant or irrelevant features are identified and removed, thereby increasing the efficiency and performance of the model.

In accordance with an embodiment, when data preprocessing module 406 processes new data for inference, data preprocessing module 406 replicates the same preprocessing steps to ensure consistency with the training data format. This helps to avoid discrepancies between the training data format and the inference data format, thereby reducing the likelihood of inaccurate or invalid model predictions.

In an embodiment, model selection module 408 includes logic for determining the most suitable algorithm or model architecture for a given dataset and problem. This module operates in part by analyzing the characteristics of the input data, such as its dimensionality, distribution, and the type of problem (classification, regression, clustering, etc.).

In an embodiment, model selection module 408 employs a variety of statistical and analytical techniques to understand data patterns, identify potential correlations, and assess the complexity of the task. Based on this analysis, it then matches the data characteristics with the strengths and weaknesses of various available models. This can range from simple linear models for less complex problems to sophisticated deep learning architectures for tasks requiring feature extraction and high-level pattern recognition, such as image and speech recognition.

In an embodiment, model selection module 408 utilizes techniques from the field of Automated ML (AutoML). AutoML systems automate the process of model selection by rapidly prototyping and evaluating multiple models. They use techniques like Bayesian optimization, genetic algorithms, or reinforcement learning to explore the model space efficiently. Model selection module 408 may use these techniques to evaluate each candidate model based on performance metrics relevant to the task. For example, accuracy, precision, recall, or F1 score may be used for classification tasks and mean squared error metrics may be used for regression tasks. Accuracy measures the proportion of correct predictions (both positive and negative). Precision measures the proportion of actual positives among the predicted positive cases. Recall (also known as sensitivity) evaluates how well the model identifies actual positives. F1 Score is a single metric that accounts for both false positives and false negatives. The mean squared error (MSE) metric may be used for regression tasks. MSE measures the average squared difference between the actual and predicted values, providing an indication of the model's accuracy. A lower MSE may indicate a model's greater accuracy in predicting values, as it represents a smaller average discrepancy between the actual and predicted values.

In accordance with an embodiment, model selection module 408 also considers computational efficiency and resource constraints. This is meant to help ensure the selected model is both accurate and practical in terms of computational and time requirements. In an embodiment, certain features of model selection module 408 are configurable such as a configured bias toward (or against) computational efficiency.

In accordance with an embodiment, training module 410 manages the ‘learning’ process of ML models by implementing various learning algorithms that enable models to identify patterns and make predictions or decisions based on input data. In an embodiment, the training process begins with the preparation of the dataset after preprocessing; this involves splitting the data into training and validation sets. The training set is used to teach the model, while the validation set is used to evaluate its performance and adjust parameters accordingly. Training module 410 handles the iterative process of feeding the training data into the model, adjusting the model's internal parameters (like weights in neural networks) through backpropagation and optimization algorithms, such as stochastic gradient descent or other algorithms providing similarly useful results.

In accordance with an embodiment, training module 410 manages overfitting, where a model learns the training data too well, including its noise and outliers, at the expense of its ability to generalize to new data. Techniques such as regularization, dropout (in neural networks), and early stopping are implemented to mitigate this. Additionally, the module employs various techniques for hyperparameter tuning; this involves adjusting model parameters that are not directly learned from the training process, such as learning rate, the number of layers in a neural network, or the number of trees in a random forest.

In an embodiment, training module 410 includes logic to handle different types of data and learning tasks. For instance, it includes different training routines for supervised learning (where the training data comes with labels) and unsupervised learning (without labeled data). In the case of deep learning models, training module 410 also manages the complexities of training neural networks that include initializing network weights, choosing activation functions, and setting up neural network layers.

In an embodiment, evaluation and tuning module 412 incorporates dynamic feedback mechanisms and facilitates continuous model evolution to help ensure the system's relevance and accuracy as the data landscape changes. Evaluation and tuning module 412 conducts a detailed evaluation of a model's performance. This process involves using statistical methods and a variety of performance metrics to analyze the model's predictions against a validation dataset. The validation dataset, distinct from the training set, is instrumental in assessing the model's predictive accuracy and its capacity to generalize beyond the training data. The module's algorithms meticulously dissect the model's output, uncovering biases, variances, and the overall effectiveness of the model in capturing the underlying patterns of the data.

In an embodiment, evaluation and tuning module 412 performs continuous model tuning by using hyperparameter optimization. Evaluation and tuning module 412 performs an exploration of the hyperparameter space using algorithms, such as grid search, random search, or more sophisticated methods like Bayesian optimization. Evaluation and tuning module 412 uses these algorithms to iteratively adjust and refine the model's hyperparameters—settings that govern the model's learning process but are not directly learned from the data—to enhance the model's performance. This tuning process helps to balance the model's complexity with its ability to generalize and attempts to avoid the pitfalls of underfitting or overfitting.

In an embodiment, evaluation and tuning module 412 integrates data feedback and updates the model. Evaluation and tuning module 412 actively collects feedback from the model's real-world applications, an indicator of the model's performance in practical scenarios. Such feedback can come from various sources depending on the nature of the application. For example, in a user-centric application like a recommendation system, feedback might comprise user interactions, preferences, and responses. In other contexts, such as predicting events, it might involve analyzing the model's prediction errors, misclassifications, or other performance metrics in live environments.

In an embodiment, feedback integration logic within evaluation and tuning module 412 integrates this feedback using a process of assimilating new data patterns, user interactions, and error trends into the system's knowledge base. The feedback integration logic uses this information to identify shifts in data trends or emergent patterns that were not present or inadequately represented in the original training dataset. Based on this analysis, the module triggers a retraining or updating cycle for the model. If the feedback suggests minor deviations or incremental changes in data patterns, the feedback integration logic may employ incremental learning strategies, fine-tuning the model with the new data while retaining its previously learned knowledge. In cases where the feedback indicates significant shifts or the emergence of new patterns, a more comprehensive model updating process may be initiated. This process might involve revisiting the model selection process, re-evaluating the suitability of the current model architecture, and/or potentially exploring alternative models or configurations that are more attuned to the new data.

In accordance with an embodiment, throughout this iterative process of feedback integration and model updating, evaluation and tuning module 412 employs version control mechanisms to track changes, modifications, and the evolution of the model, facilitating transparency and allowing for rollback if necessary. This continuous learning and adaptation cycle, driven by real-world data and feedback, helps to endure the model's ongoing effectiveness, relevance, and accuracy.

In an embodiment, inference module 414 transforms data raw data into actionable, precise, and contextually relevant predictions. In addition to processing and applying a trained model to new data, inference module 414 may also include post-processing logic that refines the raw outputs of the model into meaningful insights.

In an embodiment, inference module 414 includes classification logic that takes the probabilistic outputs of the model and converts them into definitive class labels. This process involves an analytical interpretation of the probability distribution for each class. For example, in binary classification, the classification logic may identify the class with a probability above a certain threshold, but classification logic may also consider the relative probability distribution between classes to create a more nuanced and accurate classification.

In an embodiment, inference module 414 transforms the outputs of a trained model into definitive classifications. Inference module 414 employs the underlying model as a tool to generate probabilistic outputs for each potential class. It then engages in an interpretative process to convert these probabilities into concrete class labels.

In an embodiment, when inference module 414 receives the probabilistic outputs from the model, it analyzes these probabilities to determine how they are distributed across some or every potential class. If the highest probability is not significantly greater than the others, inference module 414 may determine that there is ambiguity or interpret this as a lack of confidence displayed by the model.

In an embodiment, inference module 414 uses thresholding techniques for applications where making a definitive decision based on the highest probability might not suffice due to the critical nature of the decision. In such cases, inference module 414 assesses if the highest probability surpasses a certain confidence threshold that is predetermined based on the specific requirements of the application. If the probabilities do not meet this threshold, inference module 414 may flag the result as uncertain or defer the decision to a human expert. Inference module 414 dynamically adjusts the decision thresholds based on the sensitivity and specificity requirements of the application, subject to calibration for balancing the trade-offs between false positives and false negatives.

In accordance with an embodiment, inference module 414 contextualizes the probability distribution against the backdrop of the specific application. This involves a comparative analysis, especially in instances where multiple classes have similar probability scores, to deduce the most plausible classification. In an embodiment, inference module 414 may incorporate additional decision-making rules or contextual information to guide this analysis, ensuring that the classification aligns with the practical and contextual nuances of the application.

In regression models, where the outputs are continuous values, inference module 414 may engage in a detailed scaling process in an embodiment. Outputs, often normalized or standardized during training for optimal model performance, are rescaled back to their original range. This rescaling involves recalibration of the output values using the original data's statistical parameters, such as mean and standard deviation, ensuring that the predictions are meaningful and comparable to the real-world scales they represent.

In an embodiment, inference module 414 incorporates domain-specific adjustments into its post-processing routine. This involves tailoring the model's output to align with specific industry knowledge or contextual information. For example, in financial forecasting, inference module 414 may adjust predictions based on current market trends, economic indicators, or recent significant events, ensuring that the outputs are both statistically accurate and practically relevant.

In an embodiment, inference module 414 includes logic to handle uncertainty and ambiguity in the model's predictions. In cases where inference module 414 outputs a measure of uncertainty, such as in Bayesian inference models, inference module 414 interprets these uncertainty measures by converting probabilistic distributions or confidence intervals into a format that can be easily understood and acted upon. This provides users with both a prediction and an insight into the confidence level of that prediction. In an embodiment, inference module 414 includes mechanisms for involving human oversight or integrating the instance into a feedback loop for subsequent analysis and model refinement.

In an embodiment, inference module 414 formats the final predictions for end-user consumption. Predictions are converted into visualizations, user-friendly reports, or interactive interfaces. In some systems, like recommendation engines, inference module 414 also integrates feedback mechanisms, where user responses to the predictions are used to continually refine and improve the model, creating a dynamic, self-improving system.

### B. Example Operations of a Machine Learning System

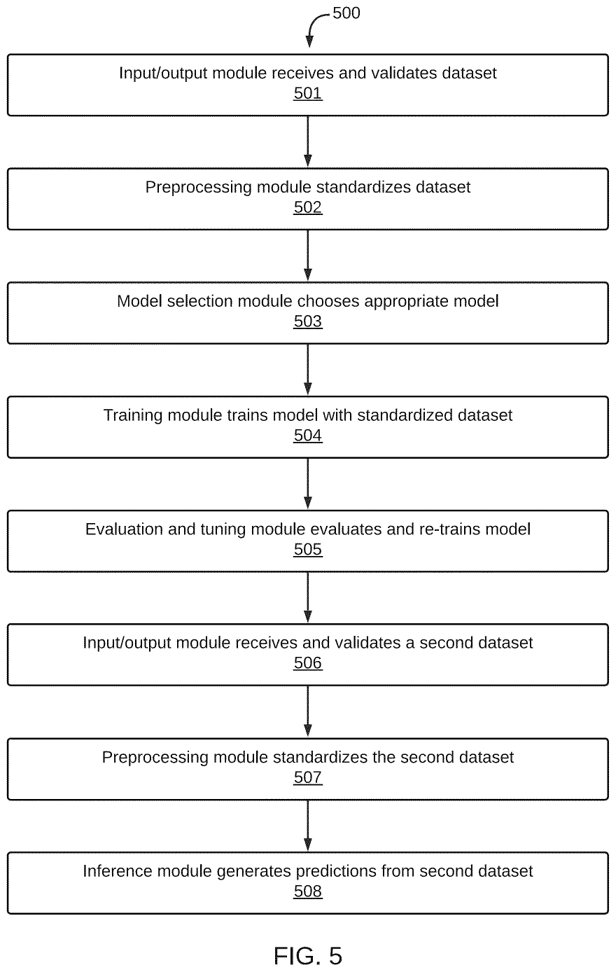

FIG. 5 illustrates example operations 500 of an ML system in one or more embodiments. In an embodiment, input/output module 404 receives a dataset intended for training (Operation 501). This data can originate from diverse sources, like databases or real-time data streams, and in varied formats, such as CSV, JSON, or XML. Input/output module 404 assesses and validates the data, ensuring its integrity by checking for consistency, data ranges, and types.

In an embodiment, training data is passed to data preprocessing module 406. Here, the data undergoes a series of transformations to standardize and clean it, making it suitable for training ML models (Operation 502). This involves normalizing numerical data, encoding categorical variables, and handling missing values through techniques like imputation.

In an embodiment, prepared data from the data preprocessing module 406 is then fed into model selection module 408 (Operation 503). This module analyzes the characteristics of the processed data, such as dimensionality and distribution, and selects the most appropriate model architecture for the given dataset and problem. It employs statistical and analytical techniques to match the data with an optimal model, ranging from simpler models for less complex tasks to more advanced architectures for intricate tasks.

In an embodiment, training module 410 trains the selected model with the prepared dataset (Operation 504). It implements learning algorithms to adjust the model's internal parameters, optimizing them to identify patterns and relationships in the training data. Training module 410 also addresses the challenge of overfitting by implementing techniques, like regularization and early stopping, ensuring the model's generalizability.

In an embodiment, evaluation and tuning module 412 evaluates the trained model's performance using the validation dataset (Operation 505). Evaluation and tuning module 412 applies various metrics to assess predictive accuracy and generalization capabilities. It then tunes the model by adjusting hyperparameters, and if needed, incorporates feedback from the model's initial deployments, retraining the model with new data patterns identified from the feedback.

In an embodiment, input/output module 404 receives a dataset intended for inference. Input/output module 404 assesses and validates the data (Operation 506).

In an embodiment, data preprocessing module 406 receives the validated dataset intended for inference (Operation 507). Data preprocessing module 406 ensures that the data format used in training is replicated for the new inference data, maintaining consistency and accuracy for the model's predictions.

In an embodiment, inference module 414 processes the new data set intended for inference, using the trained and tuned model (Operation 508). It applies the model to this data, generating raw probabilistic outputs for predictions. Inference module 414 then executes a series of post-processing steps on these outputs, such as converting probabilities to class labels in classification tasks or rescaling values in regression tasks. It contextualizes the outputs as per the application's requirements, handling any uncertainty in predictions and formatting the final outputs for end-user consumption or integration into larger systems.

In an embodiment, the ML system 400 includes an ML engine API 416. The ML engine API 416 allows for applications to leverage ML engine 402. In an embodiment, ML engine API 416 may be built on a RESTful architecture and offer stateless interactions over standard HTTP/HTTPS protocols. ML engine API 416 may feature a variety of endpoints, each tailored to a specific function within ML engine 402. In an embodiment, endpoints such as /submitData facilitate the submission of new data for processing, while /retrieveResults is designed for fetching the outcomes of data analysis or model predictions. The MLE API may also include endpoints like /updateModel for model modifications and /trainModel to initiate training with new datasets.

In an embodiment, ML engine API 416 is equipped to support SOAP-based interactions. This extension involves defining a WSDL (Web Services Description Language) document that outlines the API's operations and the structure of request and response messages. In an embodiment, ML engine API 416 supports various data formats and communication styles. In an embodiment, ML engine API 416 endpoints may handle requests in JSON format or any other suitable format. For example, ML engine API 416 may process XML, and it may also be engineered to handle more compact and efficient data formats, such as Protocol Buffers or Avro, for use in bandwidth-limited scenarios.

In an embodiment, ML engine API 416 is designed to integrate WebSocket technology for applications necessitating real-time data processing and immediate feedback. This integration enables a continuous, bi-directional communication channel for a dynamic and interactive data exchange between the application and ML engine 402.

### C. Example Generative Models of a Machine Learning System

A generative model is an ML model that is capable of generating new data instances based on the data used to train the model. A generative model may be referred to as a “generative artificial intelligence (AI) model.” Generative models learn the underlying distribution of the training data, enabling them to produce new instances of data that share properties with the original dataset. This capability makes them particularly useful in a variety of applications, including image and voice generation, text synthesis, and more sophisticated tasks like unsupervised learning, semi-supervised learning, and domain adaptation.

One type of generative model is a large language model. Large language models are designed to understand, generate, and interpret human language by processing extensive collections of data. The foundational architecture behind large language models is the transformer network, a type of neural network that excels in handling sequential data such as text. Unlike architectures, such as recurrent neural networks (RNNs) or long short-term memory networks (LSTMs), transformers do not process data in order. Instead, they leverage parallel processing to analyze entire text sequences simultaneously, significantly improving efficiency and reducing training times.

In an embodiment, a mechanism that enables transformers to handle complex language tasks is self-attention. This mechanism allows the model to weigh the importance of different words within a sentence or sequence regardless of their position. For instance, in processing the phrase “The cat sat on the mat,” the model can directly associate “cat” with “mat” without having to process the intermediate words sequentially. This ability to understand the context and relationships between words in a sentence is what makes transformer networks adept at language tasks. The self-attention mechanism assigns scores to relationships between words, highlighting the most relevant connections, so the model can focus on the most informative parts of the text.

In accordance with one or more embodiments, transformers are composed of multiple layers containing a multi-head, self-attention mechanism and a position-wise, feed-forward network. Within the architecture of transformer models, the multi-head, self-attention mechanism and position-wise, feed-forward network function in concert to process input data. The multi-head, self-attention mechanism is designed to enable parallel processing of input sequences, allowing the model to simultaneously evaluate the importance of different segments of the input relative to each other. This mechanism operates by generating multiple sets of query, key, and value vectors for each element in the input sequence through linear transformation. The relevance of each element to every other element is calculated using a scaled dot-product attention function that computes the attention scores by taking the dot product of the query vector with the key vectors, dividing each by the square root of the dimension of the key vectors to scale the scores, then applying a SoftMax function to obtain the weights for the value vectors. The scaled dot-product attention function is applied independently by each head in the multi-head self-attention mechanism. The outputs of these heads are then concatenated and linearly transformed, allowing the model to capture information from different representation subspaces.

In accordance with one or more embodiments, following the multi-head, self-attention mechanism is the position-wise, feed-forward network. This component comprises two linear transformations with a non-linear activation function in between. Each element of the input sequence, now enriched with context by the self-attention mechanism, is processed independently through the same feed-forward network. The first linear transformation increases the dimensionality of the input, allowing for a richer representation space. The non-linear activation function introduces the capability to capture non-linear relationships within the data. The second linear transformation then reduces the dimensionality back to that of the model's hidden layers, preparing the output for either further processing by subsequent layers or final output generation. This sequence of operations is applied to each position in the sequence, so the model can learn complex patterns across different parts of the input data without relying on the sequential processing inherent to previous architectures, such as RNNs or LSTMs.

In accordance with one or more embodiments, integrating these components within the transformer architecture facilitates the model's ability to understand and generate human language by leveraging both the global context provided by the self-attention mechanism and the local, position-specific transformations applied by the feed-forward networks. Through the repetitive stacking of layers, transformers achieve a depth of representation that allows for the processing of linguistic information across varying levels of complexity.

In accordance with one or more embodiments, input/output module 404, when used for large language models, handles textual data, converting input text into a format that the model can process. This typically involves tokenization, where the text is broken down into manageable pieces, such as words or sub words, and then converted into numerical representations. These representations, or embeddings, capture semantic information about the text that is then fed into the model for processing. The output from the model is converted from numerical form back into human-readable text, following the generation of predictions or responses.

In accordance with one or more embodiments, data preprocessing module 406 in the context of large language models may include steps such as normalization, where the text is converted to a uniform case and punctuation is standardized. This process ensures that the model treats similar words or symbols consistently, reducing the complexity of the input space. Additionally, techniques such as sentence segmentation may be applied to manage longer texts, enabling the model to process information in chunks that align with natural language structures.

In accordance with one or more embodiments, model selection module 408, when used for large language models involves choosing a specific architecture and configuration that is best suited to the task at hand. This decision is based on various factors, such as the size of the available training data, the complexity of the language tasks to be performed, and computational resource constraints. Models may vary in size from millions to billions of parameters, with larger models generally capable of more nuanced language understanding and generation but requiring significantly more computational power to train and operate.

In accordance with one or more embodiments, training module 410, when used for large language models, is configured to adjust the model's parameters through exposure to training data. This process utilizes optimization algorithms, such as stochastic gradient descent, to minimize the difference between the model's predictions and the actual desired outputs. The training process is computationally intensive, often requiring specialized hardware such as GPUs or TPUs to manage the large volumes of data and the complexity of the model calculations. During training, techniques, such as dropout and layer normalization, are used to improve model generalization and prevent overfitting (i.e., when a model learns the detail and noise in the training data to the extent that it negatively impacts the model's performance on new data).

In accordance with one or more embodiments, evaluation and tuning module 412 assesses the performance of large language models using metrics such as perplexity, accuracy, and F1 score, depending on the specific language tasks. Evaluation may involve comparing the model's output against a set of labeled validation data, providing insight into how well the model has learned to perform tasks, such as text classification, question answering, or text generation. Tuning involves adjusting model parameters or training strategies based on evaluation outcomes to improve performance. This may include hyperparameter tuning, where parameters that govern the training process, such as learning rate or batch size, are adjusted.

In accordance with one or more embodiments, inference module 414, in the context of large language models, is responsible for generating predictions or responses based on new, unseen data. This process involves feeding the input data through the trained model to produce an output. Inference can be used for a variety of applications, including translating text, generating human-like responses in a chatbot, or summarizing articles.

Another type of generative model is a large multimodal model (LMM). A large multimodal model is an advanced ML model capable of processing and generating data across multiple modalities, such as text, images, audio, and video. These models integrate diverse datasets during training to learn the underlying distribution of different data types, enabling them to produce outputs that reflect a comprehensive understanding of the input data. These models can be used for applications such as image captioning, text-to-image generation, image-to-text generation, visual question answering, and more, where understanding the relationship between different data types is crucial. By leveraging diverse datasets during training, large multimodal models learn to create coherent and contextually relevant outputs across various modalities, enhancing their utility in complex, real-world scenarios.

The architecture of large multimodal models combines elements from different neural network designs to handle diverse data types effectively. For example, convolutional neural networks (CNNs) are often used for processing visual data, while transformer networks handle textual data, enabling the model to extract and synthesize features from both images and text. This integration results in outputs that accurately represent the input data, reflecting a deep understanding of both modalities. The transformer architecture, known for its ability to manage sequential data, is frequently adapted to work alongside CNNs, allowing these models to benefit from the strengths of each neural network type.

In at least some instances, the self-attention mechanism, a cornerstone of transformer networks, is integral to the functioning of large multimodal models. It enables the model to weigh the importance of different elements within an input sequence, regardless of their position, allowing it to capture intricate relationships between various data types. For example, in an image captioning task, the model can associate specific visual features with corresponding descriptive text, enhancing the coherence and accuracy of the generated captions. By assigning scores to relationships between elements, the self-attention mechanism highlights the most relevant connections, enabling the model to focus on the most informative parts of the input data and perform complex multimodal tasks effectively.

In large multimodal models, data preprocessing is a step that ensures the input data is in a suitable format for the model to process. This involves tasks such as tokenization for text data, where the text is broken down into manageable pieces, and feature extraction for image data, where key visual elements are identified and encoded. By standardizing and normalizing different data types, preprocessing reduces the complexity of the input space, enabling the model to treat similar elements consistently. Effective preprocessing is essential for the model to integrate information from various modalities and produce accurate, meaningful outputs.

Training large multimodal models involves optimizing their parameters through exposure to diverse datasets that include paired data from different modalities. This computationally intensive process often requires specialized hardware like GPUs or TPUs to manage the large volumes of data and the complexity of the model calculations. Techniques such as dropout and layer normalization are employed to improve model generalization and prevent overfitting. By iteratively adjusting the model's parameters, the training process enables the model to learn underlying patterns and relationships within the data, enhancing its ability to generate coherent and contextually relevant outputs across different modalities.

Evaluation and tuning of large multimodal models are conducted using various metrics tailored to the specific tasks they are designed to perform. For example, BLEU scores are used for text generation tasks, while accuracy is commonly applied for visual recognition tasks to assess performance. Tuning involves adjusting hyperparameters and refining training strategies based on evaluation results to enhance the model's effectiveness. This iterative process ensures that the model can perform a wide range of multimodal tasks with high accuracy and relevance, making it a versatile tool for applications requiring the integration of different types of data.

Large multimodal models represent a significant advancement in ML by leveraging sophisticated architectures that combine different neural network types and apply self-attention mechanisms. This enables them to perform complex tasks that require understanding and synthesizing information from diverse data types. Effective preprocessing, rigorous training, and thorough evaluation are crucial to their success, allowing these models to generate coherent and contextually relevant outputs across a wide range of applications.

In accordance with one or more embodiments, other types of models besides large language models and large multimodal models belong to the broad category of generative models. For example, stochastic models directly incorporate randomness into their structure, making them inherently generative as they can produce a diverse set of outputs for a given input. Generative Adversarial Networks (GANs) learn to generate new data that is indistinguishable from the data they were trained on, using a dual-network architecture that involves a generative component. Variational Autoencoders (VAEs) are explicitly designed for generating new data points by learning a distribution of the input data and encode inputs into a latent space and generate outputs by sampling from this space, making them inherently generative. Sequence-to-sequence models are generative in nature when used with sampling strategies. Although this list of generative model types is not exhaustive, it illustrates the broad use of the term generative model beyond large language models.

Although generative models can be leveraged for classification tasks, they inherently operate on principles of randomness, leading to a spectrum of possible outcomes in response to identical inputs. Unlike deterministic models that yield a consistent result whenever the same input is given, generative models use the randomness in the data they are trained on to both mimic and diversify from the training data. This diversity makes generative models ideal for generating new and varied data points as well as for tasks that require creativity and novelty. However, a reliance on randomness creates a trade-off between predictability and flexibility for generative models, potentially making them less predictable in scenarios where uniform outcomes may be expected such as classification tasks.

### 7. COMPUTER NETWORKS AND CLOUD NETWORKS

In one or more embodiments, a computer network provides connectivity among a set of nodes. The nodes may be local to and/or remote from each other. The nodes are connected by a set of links. Examples of links include a coaxial cable, an unshielded twisted cable, a copper cable, an optical fiber, and a virtual link.

A subset of nodes implements the computer network. Examples of such nodes include a switch, a router, a firewall, and a network address translator (NAT). Another subset of nodes uses the computer network. Such nodes (also referred to as “hosts”) may execute a client process and/or a server process. A client process makes a request for a computing service (such as, execution of a particular application, and/or storage of a particular amount of data). A server process responds by executing the requested service and/or returning corresponding data.

A computer network may be a physical network, including physical nodes connected by physical links. A physical node is any digital device. A physical node may be a function-specific hardware device, such as a hardware switch, a hardware router, a hardware firewall, and a hardware NAT. Additionally or alternatively, a physical node may be a generic machine that is configured to execute various virtual machines and/or applications performing respective functions. A physical link is a physical medium connecting two or more physical nodes. Examples of links include a coaxial cable, an unshielded twisted cable, a copper cable, and an optical fiber.

A computer network may be an overlay network. An overlay network is a logical network implemented on top of another network (such as, a physical network). Each node in an overlay network corresponds to a respective node in the underlying network. Hence, each node in an overlay network is associated with both an overlay address (to address to the overlay node) and an underlay address (to address the underlay node that implements the overlay node). An overlay node may be a digital device and/or a software process (such as, a virtual machine, an application instance, or a thread) A link that connects overlay nodes is implemented as a tunnel through the underlying network. The overlay nodes at either end of the tunnel treat the underlying multi-hop path between them as a single logical link. Tunneling is performed through encapsulation and decapsulation.

In an embodiment, a client may be local to and/or remote from a computer network. The client may access the computer network over other computer networks, such as a private network or the Internet. The client may communicate requests to the computer network using a communications protocol, such as Hypertext Transfer Protocol (HTTP). The requests are communicated through an interface, such as a client interface (such as a web browser), a program interface, or an application programming interface (API).

In an embodiment, a computer network provides connectivity between clients and network resources. Network resources include hardware and/or software configured to execute server processes. Examples of network resources include a processor, a data storage, a virtual machine, a container, and/or a software application. Network resources are shared amongst multiple clients. Clients request computing services from a computer network independently of each other. Network resources are dynamically assigned to the requests and/or clients on an on-demand basis.

### 8. HARDWARE OVERVIEW

According to one embodiment, the techniques described herein are implemented by one or more special-purpose computing devices. The special-purpose computing devices may be hard-wired to perform the techniques, or may include digital electronic devices such as one or more application-specific integrated circuits (ASICs), field programmable gate arrays (FPGAs), or network processing units (NPUs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, FPGAs, or NPUs with custom programming to accomplish the techniques. The special-purpose computing devices may be desktop computer systems, portable computer systems, handheld devices, networking devices or any other device that incorporates hard-wired and/or program logic to implement the techniques.

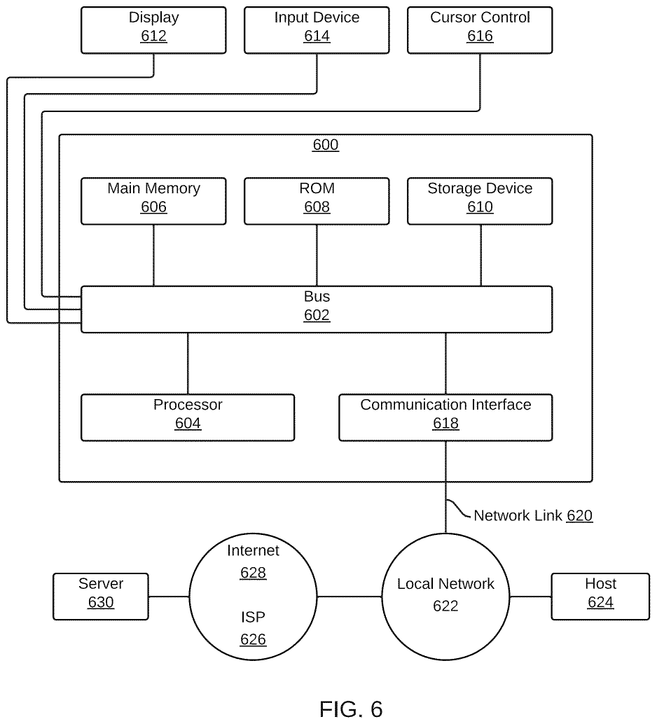

For example, FIG. 6 is a block diagram that illustrates a computer system 600 upon which an embodiment of the disclosure may be implemented. Computer system 600 includes a bus 602 or other communication mechanism for communicating information, and a hardware processor 604 coupled with bus 602 for processing information. Hardware processor 604 may be, for example, a general purpose microprocessor.

Computer system 600 also includes a main memory 606, such as a random access memory (RAM) or other dynamic storage device, coupled to bus 602 for storing information and instructions to be executed by processor 604. Main memory 606 also may be used for storing temporary variables or other intermediate information during execution of instructions to be executed by processor 604. Such instructions, when stored in non-transitory storage media accessible to processor 604, render computer system 600 into a special-purpose machine that is customized to perform the operations specified in the instructions.

Computer system 600 further includes a read-only memory (ROM) 608 or other static storage device coupled to bus 602 for storing static information and instructions for processor 604. A storage device 610, such as a magnetic disk, optical disk, or a Solid State Drive (SSD) is provided and coupled to bus 602 for storing information and instructions.

Computer system 600 may be coupled via bus 602 to a display 612, such as a cathode ray tube (CRT), for displaying information to a computer user. An input device 614, including alphanumeric and other keys, is coupled to bus 602 for communicating information and command selections to processor 604. Another type of user input device is cursor control 616, such as a mouse, a trackball, or cursor direction keys for communicating direction information and command selections to processor 604 and for controlling cursor movement on display 612. This input device typically has two degrees of freedom in two axes, a first axis (e.g., x) and a second axis (e.g., y), that allows the device to specify positions in a plane.

Computer system 600 may implement the techniques described herein using customized hard-wired logic, one or more ASICs or FPGAs, firmware and/or program logic which in combination with the computer system causes or programs computer system 600 to be a special-purpose machine. According to one embodiment, the techniques herein are performed by computer system 600 in response to processor 604 executing one or more sequences of one or more instructions contained in main memory 606. Such instructions may be read into main memory 606 from another storage medium, such as storage device 610. Execution of the sequences of instructions contained in main memory 606 causes processor 604 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage device 610. Volatile media includes dynamic memory, such as main memory 606. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, content-addressable memory (CAM), and ternary content-addressable memory (TCAM).

Storage media is distinct from but may be used in conjunction with transmission media. Transmission media participates in transferring information between storage media. For example, transmission media includes coaxial cables, copper wire and fiber optics, including the wires that comprise bus 602. Transmission media can also take the form of acoustic or light waves, such as those generated during radio-wave and infra-red data communications.

Various forms of media may be involved in carrying one or more sequences of one or more instructions to processor 604 for execution. For example, the instructions may initially be carried on a magnetic disk or solid state drive of a remote computer. The remote computer can load the instructions into its dynamic memory and send the instructions over a telephone line using a modem. A modem local to computer system 600 can receive the data on the telephone line and use an infra-red transmitter to convert the data to an infra-red signal. An infra-red detector can receive the data carried in the infra-red signal and appropriate circuitry can place the data on bus 602. Bus 602 carries the data to main memory 606, from which processor 604 retrieves and executes the instructions. The instructions received by main memory 606 may optionally be stored on storage device 610 either before or after execution by processor 604.

Computer system 600 also includes a communication interface 618 coupled to bus 602. Communication interface 618 provides a two-way data communication coupling to a network link 620 that is connected to a local network 622. For example, communication interface 618 may be an integrated services digital network (ISDN) card, cable modem, satellite modem, or a modem to provide a data communication connection to a corresponding type of telephone line. As another example, communication interface 618 may be a local area network (LAN) card to provide a data communication connection to a compatible LAN. Wireless links may also be implemented. In any such implementation, communication interface 618 sends and receives electrical, electromagnetic or optical signals that carry digital data streams representing various types of information.

Network link 620 typically provides data communication through one or more networks to other data devices. For example, network link 620 may provide a connection through local network 622 to a host computer 624 or to data equipment operated by an Internet Service Provider (ISP) 626. ISP 626 in turn provides data communication services through the world wide packet data communication network now commonly referred to as the “Internet” 628. Local network 622 and Internet 628 both use electrical, electromagnetic or optical signals that carry digital data streams. The signals through the various networks and the signals on network link 620 and through communication interface 618, which carry the digital data to and from computer system 600, are example forms of transmission media.

Computer system 600 can send messages and receive data, including program code, through the network(s), network link 620 and communication interface 618. In the Internet example, a server 630 might transmit a requested code for an application program through Internet 628, ISP 626, local network 622 and communication interface 618.

The received code may be executed by processor 604 as it is received, and/or stored in storage device 610, or other non-volatile storage for later execution.

### 9. MISCELLANEOUS; EXTENSIONS

Unless otherwise defined, all terms (including technical and scientific terms) are to be given their ordinary and customary meaning to a person of ordinary skill in the art, and are not to be limited to a special or customized meaning unless expressly so defined herein.

This application may include references to certain trademarks. Although the use of trademarks is permissible in patent applications, the proprietary nature of the marks should be respected, and every effort made to prevent their use in any manner which might adversely affect their validity as trademarks.

Embodiments are directed to a system with one or more devices that include a hardware processor and that are configured to perform any of the operations described herein and/or recited in any of the claims below.

In an embodiment, one or more non-transitory computer-readable storage media comprises instructions which, when executed by one or more hardware processors, cause performance of any of the operations described herein and/or recited in any of the claims.

In an embodiment, a method comprises operations described herein and/or recited in any of the claims, the method being executed by at least one device including a hardware processor.

Any combination of the features and functionalities described herein may be used in accordance with one or more embodiments. In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the disclosure, and what is intended by the applicants to be the scope of the disclosure, is the literal and equivalent scope of the set of claims that issue from this application, in the specific form in which such claims issue, including any subsequent correction.

What is claimed is:

1. A method, comprising:
determining that a trigger condition is satisfied for requesting a knowledge-adapted content element for augmenting a display of a set of information on a user interface, the knowledge-adapted content element based on a knowledge classification of a user with respect to a target concept associated with the set of information;
responsive to determining that the trigger condition is satisfied, generating in real-time an input prompt element for requesting the knowledge-adapted content element;
directing the input prompt element to a machine learning model to initiate execution of an inference by the machine learning model based on the input prompt element, the inference comprising generating the knowledge-adapted content element based on the knowledge classification of the user with respect to the target concept associated with the set of information, the knowledge-adapted content element comprising machine-generated content pertaining to the target concept;
receiving the knowledge-adapted content element from the machine learning model in response to directing the input prompt element to the machine learning model;
augmenting the set of information, wherein augmenting the set of information comprises displaying the machine-generated content on the user interface concurrently with displaying the set of information;
wherein the method is performed by at least one device including a hardware processor.

2. The method of claim 1,
wherein determining the knowledge classification of the user comprises:
generating a predicted user comprehension of the target concept based at least in part on the target concept and a set of contextual user information associated with the user;
wherein generating the knowledge-adapted content element comprising the machine-generated content pertaining to the target concept comprises:
generating a first natural language explanation of the target concept in context with the set of information based on the predicted user comprehension of the target concept.

3. The method of claim 2, wherein generating the knowledge-adapted content element comprising the machine-generated content pertaining to the target concept further comprises:
generating a second natural language explanation of the predicted user comprehension of the target concept; and
generating the first natural language explanation of the target concept based at least in part on the second natural language explanation of the predicted user comprehension of the target concept.

4. The method of claim 1, wherein generating the knowledge-adapted content element comprising the machine-generated content pertaining to the target concept comprises at least one of:
generating grounding information corresponding to the knowledge classification of the user, the grounding information comprising at least one of: a vocabulary set associated with the target concept for use in the machine-generated content, a length guideline for a length of the machine-generated content; or a formatting guideline for formatting the machine-generated content; and
directing a model input to a language model, the model input comprising the target concept and the grounding information, wherein the language model generates an explanation of the target concept adapted to the knowledge classification of the user based at least in part on the grounding information;
receiving the knowledge-adapted content element from the language model in response to the model input, wherein the machine-generated content of the knowledge-adapted content element comprises the explanation of the target concept.

5. The method of claim 1, further comprising:
accessing a set of health information in an electronic health record associated with a diagnosis of the user; and
determining the target concept based at least in part on the diagnosis of the user, wherein the target concept comprises a topic pertaining to improving user understanding of the diagnosis;
wherein generating the knowledge-adapted content element comprising the machine-generated content pertaining to the target concept comprises:
determining, based on the knowledge classification of the user with respect to the target concept, an example explanation of an example topic adapted to the knowledge classification of the user;
generating an explanation of the target concept based at least in part on the example explanation.

6. The method of claim 1, further comprising:
detecting an interaction with an interface element of the user interface;
responsive to detecting the interaction with the interface element:
identifying a mapping between the interface element and electronic health record comprising a set of health information associated with the user;
accessing the set of health information in an electronic records repository;
wherein determining that the trigger condition for requesting the knowledge-adapted content element is satisfied comprises:
directing a model input comprising the set of health information to a language model, wherein the language model is trained to identify particular concepts based on sets of health information that, when explained according to different knowledge classifications of different users, at least partially contribute to improving user understanding;
receiving a model response comprising the target concept from the language model in response to the model input, wherein the trigger condition comprises receiving the model response;
wherein generating the knowledge-adapted content element comprising the machine-generated content pertaining to the target concept comprises:
generating an explanation of the target concept adapted to the knowledge classification of the user; and
combining the explanation of the target concept with at least a portion of the set of health information;
wherein augmenting the set of information comprises displaying the explanation of the target concept with at least the portion of the set of health information.

7. The method of claim 1, further comprising:
detecting an interaction with an interface element of the user interface, wherein the interaction with the interface element comprises: receiving a query;
wherein determining that the trigger condition for requesting the knowledge-adapted content element is satisfied comprises:
directing a model input comprising the query to a language model, wherein the language model is trained to identify particular concepts based on queries that, when explained according to different knowledge classifications of different users, at least partially contribute to improving user understanding;
receiving a model response comprising the target concept from the language model in response to the model input, wherein the trigger condition comprises receiving the model response;
wherein generating the knowledge-adapted content element comprising the machine-generated content pertaining to the target concept comprises:
receiving a query response in response to the query;
generating an explanation of the target concept adapted to the knowledge classification of the user; and
combining the explanation of the target concept with at least a portion of the query response;
wherein augmenting the set of information comprises displaying the explanation of the target concept with at least the portion of the query response.

8. The method of claim 1, further comprising:
detecting an interaction with an interface element of the user interface, wherein the interaction with the interface element comprises: receiving a query;
wherein determining that the trigger condition for requesting the knowledge-adapted content element is satisfied comprises:
receiving a query response in response to the query;
directing a model input comprising the query response to a language model, wherein the language model is trained to identify particular concepts based on query responses that, when explained according to different knowledge classifications of different users, at least partially contribute to improving user understanding;
receiving a model response comprising the target concept from the language model in response to the model input, wherein the trigger condition comprises receiving the model response;
wherein generating the knowledge-adapted content element comprising the machine-generated content pertaining to the target concept comprises:
generating an explanation of the target concept adapted to the knowledge classification of the user; and
combining the explanation of the target concept with at least a portion of the query response;
wherein augmenting the set of information comprises displaying the explanation of the target concept with at least a portion of the query response.

9. The method of claim 1, further comprising:
detecting an interaction with an interface element of the user interface, wherein the interaction with the interface element comprises: accessing a visit summary interface for displaying visit summaries for healthcare services provided to user;
wherein determining that the trigger condition for requesting the knowledge-adapted content element is satisfied comprises:
extracting a set of health information from one or more electronic health records of the user;
directing a model input comprising the set of health information to a language model, wherein the language model is trained to identify particular concepts based on sets of health information that, when explained according to different knowledge classifications of different users, at least partially contribute to improving user understanding;
receiving a model response comprising the target concept from the language model in response to the model input, wherein the trigger condition comprises receiving the model response;
wherein generating the knowledge-adapted content element comprising the machine-generated content pertaining to the target concept comprises:
generating an explanation of the target concept adapted to the knowledge classification of the user; and
combining the explanation of the target concept with at least a portion of the set of health information to generate a visit summary;
wherein augmenting the set of information comprises displaying the visit summary on the visit summary interface.

10. The method of claim 1, wherein determining the knowledge classification of the user comprises at least one of:
determining, based at least in part on a query submitted by the user pertaining to the set of information, that a comprehension score mapped to the target concept satisfies a comprehension threshold, wherein the comprehension score represents a level of comprehension of the user with respect to the target concept;
determining that the set of information comprises a record date that satisfies a date threshold, wherein the date threshold represents a time period for the user to gain familiarity with the target concept;
determining that a complexity score mapped to the target concept satisfies a complexity threshold, wherein the complexity score represents a level of complexity of the target concept; or
determining that an encounter score mapped to the target concept satisfies an encounter threshold, wherein the encounter score represents one or more previous interactions with the user interface pertaining to the target concept.

11. One or more non-transitory computer-readable media storing program instructions that, when executed by one or more hardware processors, cause performance of operations comprising:
determining that a trigger condition is satisfied for requesting a knowledge-adapted content element for augmenting a display of a set of information on a user interface, the knowledge-adapted content element based on a knowledge classification of a user with respect to a target concept associated with the set of information;
responsive to determining that the trigger condition is satisfied, generating in real-time an input prompt element for requesting the knowledge-adapted content element;
directing the input prompt element to a machine learning model to initiate execution of an inference by the machine learning model based on the input prompt element, the inference comprising generating the knowledge-adapted content element based on the knowledge classification of the user with respect to the target concept associated with the set of information, the knowledge-adapted content element comprising machine-generated content pertaining to the target concept;
receiving the knowledge-adapted content element from the machine learning model in response to directing the input prompt element to the machine learning model;
augmenting the set of information, wherein augmenting the set of information comprises displaying the machine-generated content on the user interface concurrently with displaying the set of information.

12. The one or more non-transitory computer-readable media of claim 11, wherein determining the knowledge classification of the user comprises:
generating a predicted user comprehension of the target concept based at least in part on the target concept and a set of contextual user information associated with the user;
wherein generating the knowledge-adapted content element comprising the machine-generated content pertaining to the target concept comprises:
generating a first natural language explanation of the target concept in context with the set of information based on the predicted user comprehension of the target concept.

13. The one or more non-transitory computer-readable media of claim 11, wherein generating the knowledge-adapted content element comprising the machine-generated content pertaining to the target concept comprises at least one of:
generating grounding information corresponding to the knowledge classification of the user, the grounding information comprising at least one of: a vocabulary set associated with the target concept for use in the machine-generated content, a length guideline for a length of the machine-generated content; or a formatting guideline for formatting the machine-generated content; and
directing a model input to a language model, the model input comprising the target concept and the grounding information, wherein the language model generates an explanation of the target concept adapted to the knowledge classification of the user based at least in part on the grounding information;
receiving the knowledge-adapted content element from the language model in response to the model input, wherein the machine-generated content of the knowledge-adapted content element comprises the explanation of the target concept.

14. The one or more non-transitory computer-readable media of claim 11, wherein the operations further comprise:
accessing a set of health information in an electronic health record associated with a diagnosis of the user; and
determining the target concept based at least in part on the diagnosis of the user, wherein the target concept comprises a topic pertaining to improving user understanding the diagnosis;
wherein generating the knowledge-adapted content element comprising the machine-generated content pertaining to the target concept comprises:
determining, based on the knowledge classification of the user with respect to the target concept, an example explanation of an example topic adapted to the knowledge classification of the user;
generating an explanation of the target concept based at least in part on the example explanation.

15. The one or more non-transitory computer-readable media of claim 11, wherein the operations further comprise:
detecting an interaction with an interface element of the user interface;
responsive to detecting the interaction with the interface element:
identifying a mapping between the interface element and electronic health record comprising a set of health information associated with the user;
accessing the set of health information in an electronic records repository;
wherein determining that the trigger condition for requesting the knowledge-adapted content element is satisfied comprises:
directing a model input comprising the set of health information to a language model, wherein the language model is trained to identify particular concepts based on sets of health information that, when explained according to different knowledge classifications of different users, at least partially contribute to improving user understanding;
receiving a model response comprising the target concept from the language model in response to the model input, wherein the trigger condition comprises receiving the model response;
wherein generating the knowledge-adapted content element comprising the machine-generated content pertaining to the target concept comprises:
generating an explanation of the target concept adapted to the knowledge classification of the user; and
combining the explanation of the target concept with at least a portion of the set of health information;
wherein augmenting the set of information comprises displaying the explanation of the target concept with at least the portion of the set of health information.

16. The one or more non-transitory computer-readable media of claim 11, wherein the operations further comprise:
detecting an interaction with an interface element of the user interface, wherein the interaction with the interface element comprises: receiving a query;
wherein determining that the trigger condition for requesting the knowledge-adapted content element is satisfied comprises:
directing a model input comprising the query to a language model, wherein the language model is trained to identify particular concepts based on queries that, when explained according to different knowledge classifications of different users, at least partially contribute to improving user understanding;
receiving a model response comprising the target concept from the language model in response to the model input, wherein the trigger condition comprises receiving the model response;
wherein generating the knowledge-adapted content element comprising the machine-generated content pertaining to the target concept comprises:
receiving a query response in response to the query;
generating an explanation of the target concept adapted to the knowledge classification of the user; and
combining the explanation of the target concept with at least a portion of the query response;
wherein augmenting the set of information comprises displaying the explanation of the target concept with at least the portion of the query response.

17. The one or more non-transitory computer-readable media of claim 11, wherein the operations further comprise:
detecting an interaction with an interface element of the user interface, wherein the interaction with the interface element comprises: receiving a query;
wherein determining that the trigger condition for requesting the knowledge-adapted content element is satisfied comprises:
receiving a query response in response to the query;
directing a model input comprising the query response to a language model, wherein the language model is trained to identify particular concepts based on query responses that, when explained according to different knowledge classifications of different users, at least partially contribute to improving user understanding;
receiving a model response comprising the target concept from the language model in response to the model input, wherein the trigger condition comprises receiving the model response;
wherein generating the knowledge-adapted content element comprising the machine-generated content pertaining to the target concept comprises:
generating an explanation of the target concept adapted to the knowledge classification of the user; and
combining the explanation of the target concept with at least a portion of the query response;
wherein augmenting the set of information comprises displaying the explanation of the target concept with at least a portion of the query response.

18. The one or more non-transitory computer-readable media of claim 11, wherein the operations further comprise:
detecting an interaction with an interface element of the user interface, wherein the interaction with the interface element comprises: accessing a visit summary interface for displaying visit summaries for healthcare services provided to user;
wherein determining that the trigger condition for requesting the knowledge-adapted content element is satisfied comprises:
extracting a set of health information from one or more electronic health records of the user;
directing a model input comprising the set of health information to a language model, wherein the language model is trained to identify particular concepts based on sets of health information that, when explained according to different knowledge classifications of different users, at least partially contribute to improving user understanding;
receiving a model response comprising the target concept from the language model in response to the model input, wherein the trigger condition comprises receiving the model response;
wherein generating the knowledge-adapted content element comprising the machine-generated content pertaining to the target concept comprises:
generating an explanation of the target concept adapted to the knowledge classification of the user; and
combining the explanation of the target concept with at least a portion of the set of health information to generate a visit summary;
wherein augmenting the set of information comprises displaying the visit summary on the visit summary interface.

19. The one or more non-transitory computer-readable media of claim 11, wherein determining the knowledge classification of the user comprises at least one of:
determining, based at least in part on a query submitted by the user pertaining to the set of information, that a comprehension score mapped to the target concept satisfies a comprehension threshold, wherein the comprehension score represents a level of comprehension of the user with respect to the target concept;
determining that the set of information comprises a record date that satisfies a date threshold, wherein the date threshold represents a time period for the user to gain familiarity with the target concept;
determining that a complexity score mapped to the target concept satisfies a complexity threshold, wherein the complexity score represents a level of complexity of the target concept; or
determining that an encounter score mapped to the target concept satisfies an encounter threshold, wherein the encounter score represents one or more previous interactions with the user interface pertaining to the target concept.

20. A system comprising:
one or more hardware processors;
one or more non-transitory computer-readable media; and
program instructions stored on the one or more non-transitory computer-readable media that, when executed by the one or more hardware processors, cause the system to perform operations comprising:
determining that a trigger condition is satisfied for requesting a knowledge-adapted content element for augmenting a display of a set of information on a user interface, the knowledge-adapted content element based on a knowledge classification of a user with respect to a target concept associated with the set of information;
responsive to determining that the trigger condition is satisfied, generating in real-time an input prompt element for requesting the knowledge-adapted content element;
directing the input prompt element to a machine learning model to initiate execution of an inference by the machine learning model based on the input prompt element, the inference comprising generating the knowledge-adapted content element based on the knowledge classification of the user with respect to the target concept associated with the set of information, the knowledge-adapted content element comprising machine-generated content pertaining to the target concept;
receiving the knowledge-adapted content element from the machine learning model in response to directing the input prompt element to the machine learning model;
augmenting the set of information, wherein augmenting the set of information comprises displaying the machine-generated content on the user interface concurrently with displaying the set of information.
